(19) United States
(12) Patent Application Publication
(10) Pub. No.: US 2003/0140370 A1
(43) Pub. Date: Jul. 24, 2003

(54) Fungal glyoxal oxidases

(76) Inventors: Christian Aichinger, Koln (DE); Peter Schreier, Koln (DE); Birgitta Leuthner, Langenfeld (DE); Martin Adamczewski, Koln (DE); Stefan Hillebrand, Neuss (DE); Karl-Heinz Kuck, Langenfeld (DE); J.A.L. van Kan, Rhenen (NL); Jaap Visser, Wageningen (NL); Francesca L. Stefanato, Zurich (CH); Regine Kahmann, Marburg (DE); Michael Bolker, Marburg (DE)

Correspondence Address: BAYER CROPSCIENCE LP, 100 BAYER ROAD, PITTSBURGH, PA, 15205, US

(21) Appl. No.: 10/242,576

(22) Filed: Sep. 12, 2002

(30) Foreign Application Priority Data
Sep. 13, 2001 (DE) 101 45 095.8
Dec. 4, 2001 (DE) 101 59 375.9
May 16, 2002 (DE) 102 21 725.4

(51) Int. Cl. A01H 1/00; C07H 21/04; C12Q 1/18; C12N 9/24; C12N 1/16; C12N 15/82; C12P 21/02

(52) U.S. Cl. 800/279; 435/32; 435/69.1; 435/254.1; 435/200; 435/320.1; 435/419; 536/23.2

(57) ABSTRACT

The invention relates to methods for identifying fungicides, to nucleic acids which encode fungal polypeptides with the biological activity of glyoxal oxidases, to the polypeptides encoded by them, to their use as targets for fungicides, their use for identifying new fungicidally active compounds, to methods for finding modulators of these polypeptides, and to transgenic organisms containing these polyypeptides.

The invention relates to methods for identifying fungicides and to nucleic acids which encode fungal polypeptides with the biological activity of glyoxal oxidases, to the polypeptides encoded by them, and to their use as targets for fungicides and their use for identifying new fungicidally active compounds, and to methods of finding modulators of these polypeptides, and, finally, to transgenic organisms containing sequences encoding fungal polypeptides with the function of a glyoxal oxidase.

Undesired fungal growth which leads every year to considerable damage, for example in agriculture, can be controlled by the use of fungicides. The demands made on fungicides have increased constantly with regard to their activity, their costs and especially ecological soundness. There exists therefore a demand for novel substances or classes of substances which can be developed into potent and ecologically sound novel fungicides. In general, it is necessary to search for such novel lead structures in greenhouse tests. However, such tests require a high input of labour and a high financial input. The number of the substances which can be tested in the greenhouse is, accordingly, limited. An alternative to such tests is the use of what are known as high-throughput screening methods (HTS). This involves testing a large number of individual substances with regard to their effect on cells, individual gene products or genes in an automated method. When certain substances are found to have an effect, they can be studied in conventional screening methods and, if appropriate, developed further.

Advantageous targets for fungicides are frequently searched for in essential biosynthesis pathways. Ideal fungicides are, moreover, those substances which inhibit gene products which have a decisive importance in the manifestation of the pathogenicity of a fungus. An example of such a fungicide is, for example, the active substance carpropamid, which inhibits fungal melanin biosynthesis and thus prevents the formation of intact appressoria (adhesion organs). However, there is only a very small number of known gene products which play such a role for fungi. Moreover, fungicides are known which lead to auxotrophism of the target cells by inhibiting corresponding biosynthesis pathways and, as a consequence, to the loss of pathogenicity. Thus, for example, the inhibition of adenosin deaminase upon addition of ethirimol leads to a significantly reduced pathogenicity in Blumeria graminis (Hollomon, D. W. 1979).

The fungus Phanerochaete chrysosporium, which belongs to the Basidiomycetes, is capable of degrading wood lignin under deficiency conditions. This degradation occurs enzymatically by the manganese-dependent lignin peroxidases (MnPs) and lignin peroxidases (LiPs). Hydrogen peroxide (H₂O₂) acts as substrate for these enzymes (Kersten et al., 1990). The hydrogen peroxide is provided by a glyoxal oxidase which catalyses the following reaction:

RCHO+O₂+H₂O.RCO₂H+H₂O₂

In this reaction, an aldehyde function is oxidized to the carboxylic acid while reducing elemental oxygen to hydrogen peroxide. The substrate specificity of the enzyme is broad so that a series of simple aldehydes, α-dicarbonyl compounds and various α-hydroxycarbonyl compounds such as, for example, HCHO, CH₃CHO, CH₂OHCHO, CHOCHO, CHOCOOH, CH₂OHCOCH₂OH, CHOCHOHCH₂OH or else CH₃COCHO are accepted as substrate. In addition, other products of the conversion of lignin model substances by lignin peroxidase are also converted by glyoxal oxidase (Kersten et al., 1995), but in particular glyoxal and methylglyoxal as intermediate metabolites in the case of growth on the main components of lignocellulose (Kersten et al., 1993). Apart from the ability of the fungus Phanerochaete chrysosporium to degrade lignin by means of glyoxal oxidase, nothing has been known about another function which the enzyme exerts for the fungus.

The Phanerochaete chrysosporium glyoxal oxidase is a copper metalloenzyme which constitutes an essential component of the lignin biodegradation pathway (Whittaker et al., 1996). The enzyme is secreted. Glyoxal oxidase firstly provides hydrogen 5 peroxide for peroxidases and, secondly, converts methylglyoxal and glyoxal, which are found as secondary metabolites in the medium of lignolytic cultures, as main substrates (Kersten et al., 1987).

Spectroscopic studies have demonstrated that an unusual free radical, which is bound to the copper ion, is present in the active centre, as is the case in the fungal metalloenzyme galactose oxidase. A homology comparison between the Phanerochaete chrysosporium glyoxal oxidase and the U. maydis glyoxal oxidase 1 (Glo 1) according to the invention (see FIG. 1) and also the B. cinerea glyoxal oxidase permits the U. maydis enzyme to be assigned to the enzyme class of what are known as the radical copper oxidases. In this enzyme class, the catalytic motif is formed by an amino side chain which has the radical attached to it and which is bound to the copper ion (formula I).

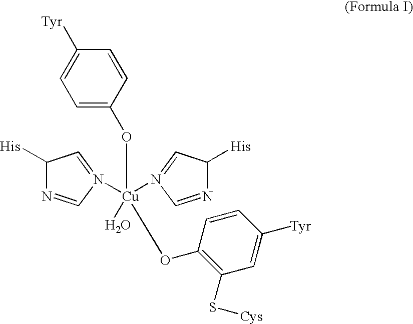

Finally, a sequence alignment of galactose oxidase and Phanerochaete glyoxal oxidase, followed by site-directed mutagenesis (Whittaker et al., 1999) allowed the other catalytically important amino acids to be assigned. EPR-spectroscopic studies identified two nitrogen ligands in a copper(II) complex, and absorption and raman spectroscopy identified the tyrosine and the tyrosine-cysteine dimer ligand in the active centre. These amino acids were the following amino acids and their positions:

Tyrosine ligand 1: Tyr 178 (U. maydis) and Tyr 273 (B. cinerea),

Tyrosine ligand 2: Tyr 452 (U. maydis) and Tyr 499 (B. cinerea),

Histidine ligand 1: His 453 (U. maydis) and His 500 (B. cinerea),

Histidine ligand 2: His 555 (U. maydis) and His 597 (B. cinerea),

Cysteine residue: Cys 105 (U. maydis) and Cys 209 (B. cinerea).

These conserved amino acids, which are characteristic for the Cu²⁺ ion bond and which are present in all polypeptides according to the invention, are thus a structurally characteristic feature of these enzymes. In contrast to other radical enzymes, which catalyse the processes while transferring one electron, two electrons are transferred by this catalytic centre. The enzyme from the class of the radical copper oxidases which has been studied most thoroughly is galactose oxidase, whose crystal structure has also been elucidated.

Glyoxal oxidases from fungal organisms other than Phanerochaete chrysosporium are as yet unknown.

Complete cDNA clones and the corresponding genes (genomic sequences or cDNA sequences) encoding for glyoxal oxidase have now been isolated from Ustilago maydis and from Botrytis cinerea within the present invention.

The smut fungus Ustilago maydis, a Basidiomycete, attacks maize plants. The disease occurs in all areas where maize is grown, but gains importance only during dry years. Typical symptoms are the gall-like, fist-sized swellings (blisters) which are formed on all aerial plant parts. The galls are first covered by a whitish-grey coarse membrane. When the membrane ruptures, a black mass of ustilospores, which is first greasy and later powdery, is released. Further species of the genus Ustilago are, for example, U. nuda (causes loose smut of barley and wheat), U. nigra (causes black smut of barley), U. hordei (causes covered smut of barley) and U. avenae (causes loose smut of oats).

The fungus Botrytis cinerea, an Ascomycete, causes what is known as “grey mould”. This is the disease which consistently causes severe damage in agriculture and is therefore controlled vigorously. It is capable of infecting all parts of the plant, but is particularly damaging to maturing berries. The cosmopolitan fungus is omnivorous and survives as a saprophyte on wood and plant residues or else as a mycelium or as sclerotia. It penetrates through wounds, but is also capable of infecting the plant post-anthesis via flower residues. It is latent in green berries; it is only after maturation has started that its development is fulminant.

Knock-out mutants have now been produced both in U. maydis and in B. cinerea with the aid of the abovementioned genomic DNA or its fragments; surprisingly, they led to apathogenicity of the fungi in both cases, that is to say in a Basidiomycete and in an Ascomycete, both of which are plant-pathogenic. It must be noted that three different genes, viz. glo1, glo2 and glo3, all of which encode a glyoxal oxidase, can be identified in Ustilago maydis. It has been found in the context of the present invention that the above-described effect is obtained in the case of the gene glo1 (cf. SEQ ID NO: 1 and 3), while the knock-out of glo2, in contrast, has no effect on the pathogenicity of the fungus. glo3, like glo1, was identified as a mutant during an apathogenicity screening as pathogenicity determinant. The reason for these different phenotypes may be identified in the expression pattern of the different enzymes, in their cellular localization, or else in the specific activity of the enzymes. Obviously, however, it is precisely glo1 which plays a decisive role in the pathogenicity of the fungus.

Morphologically noticeable mutants of strain CL13 have already been isolated (M. Bölker and R. Kahmann, unpublished) in an REMI mutagenesis approach (restriction enzyme mediated integration, see, for example, Kahmann and Basse 1999). The REMI mutant #5662 is distinguished by a flaky, matted phenotype. In addition, the mutant shows noticeable melanization.

No infection of maize plants was detected in a pathogenicity test, that is to say that the mutant is apathogenic. Plasmid rescue experiments were carried out to obtain the nucleic acids encoding glyoxal oxidase.

It has now been possible within the scope of the present invention to reisolate, by a plasmid rescue experiment (see Example 1), those sequences which flank the insertion site. In this manner, the sequences encoding glyoxal oxidase, in this case glo1, are isolated. In this context, sequencing revealed that the insertion had taken place 770 bp downstream of the start codon for putative ORF. Its deduced amino acid sequence shows similarity with the Phanerochaete chrysosporium glyoxal oxidase. The Ustilago gene was termed glo1 (glyoxal oxidase 1). Since the correlation of an REMI insertion with the observed phenotype of the mutants is not always successful, the glo1 gene in the two haploid strains Um518 and Um521 was additionally deleted for the purposes of the present invention in order to establish an unambiguous relationship between phenotype and gene (see Example 2). First, a 1151 bp and a 1249 bp DNA fragment 5′ and 3′, respectively, of the putative glo1 ORF were amplified by PCR. The fragments were subsequently cleaved with the restriction enzyme SfiI and ligated with the SfiI-cleaved hygromycin B cassette (1884 bp fragment from pBS-hhn) such that 1931 nucleotides were deleted from the ORF of the glo1 gene (see FIG. 2B and Kämper and Schreier, 2001). This knock-out cassette was likewise amplified by PCR (see Example 2). In the case of a homologous recombination, the N-terminal portion of glo1 is thus replaced by the hygromycin B cassette. The zero mutants were selected by Southern analysis of the transformants with a glo1-specific DNA probe (see FIG. 2A). It emerged that eight out of 10 transformants showed the expected restriction pattern in the Southern analysis. The strains 518Δglo1#1, 518 Δglo1#4 or 521 Δglo1#7 and 521 Δglo1#9 were chosen for further analyses.

As can be seen from FIG. 4, the glo1 zero mutants exhibit a pleiotropic morphological defect. Thus, handling of the glo1 zero mutants also demonstrates that the cells, when grown on plate media, adhere considerably less with each other in comparison with wild-type strains. In order to characterize this phenotype in greater detail, studies, for example microscopic studies, can be carried out. To this end, cells are applied to slides and observed in a differential interference contrast microscope (FIG. 4). It emerges that the cells are elongated in comparison with wild-type strains. Moreover, increased vacuolization can be observed. Moreover, the cytokinesis of mutant cells is adversely affected, and the increased development of septa is observed (see also FIG. 3). Cells which are globular in shape and which are located in the centre of unseparated cell aggregations are also noticeable. In summary, all the signs of a pleiotropic morphological defect are observed in the zero mutants according to the invention.

Furthermore, it must also be noted that mixtures of compatible glo1 zero mutants are apathogenic. To study the effect of the glo1 zero-allele on pathogenicity, plant infections were thus carried out for the purposes of the present invention. To this end, in each case two independent compatible glo1 zero mutants were grown, washed and mixed. The mixtures were then injected into young maize plants. For comparison, maize plants were infected with mixtures of compatible wild-type strains (Um518 and Um521). While tumour formation was already observed after one week in the control experiment, no symptoms whatsoever were found in the mixture of compatible mutants. Two weeks post-infection, 97 out of 102 infected plants in the control infection had formed tumours. Three more plants showed the anthocyanin hue, which is typical of fungal infections. Thus, 100 out of 102 infected plants (98%) showed symptoms of pathogenicity (see Table I). In the case of infections with mixtures of compatible mutants, neither tumour formation nor anthocyanin hues were observed (see Table I). This means that compatible zero mutants of glo1 are not capable of infecting maize plants, that is to say their pathogenicity is defective.

TABLE I
Mixtures of compatible glo1 zero mutants

| | Σ plants | Tumour | Anthocyanin | Σ symptoms | Pathogenicity (%) |
|---|---|---|---|---|---|
| Um 518 × Um 521 | 102 | 97 | 3 | 100 | 98 |
| 518Δglo1—1 × 521Δglo1-7 | 101 | 0 | 0 | 0 | 0 |
| 518Δglo1-4 × 521Δglo1-9 | 106 | 0 | 0 | 0 | 0 |

It is furthermore noticeable that the mating behaviour of the glo1 zero mutants is limited. Thus, the formation of dikaryotic filaments in mixtures of compatible glo1 mutant strains can no longer be observed. When crossing mutants with compatible wild-types, a residual activity with regard to the mating behaviour can be observed in respect to the formation of dikaryons (see FIG. 4), which allows the conclusion that cell fusion is defective.

The study of corresponding knock-out mutants in B. cinerea gave completely analogous results. Again, it was demonstrated clearly that disruption of the gene which encodes glyoxal oxidase leads to defective pathogenicity in B. cinerea (see Example 9 and FIGS. 9 to 12).

It was therefore concluded from these results that glyoxal oxidase plays a particular role in developing pathogenicity, not only in the case of one specific fungus, but in the case of phytopathogenic fungi per se. The importance of glyoxal oxidase for pathogenicity, viability in the host and the life cycle of the phytopathogenic fungi was thus recognized for the first time and for the first time identified as an optimal target for the search for novel, specific fungicides. The possibility of identifying, with the aid of this target, lead structures which may be entirely new has thus been provided for the first time. New fungicides can thus be provided starting from such compounds which inhibit glyoxal oxidase.

Furthermore provided by means of the genomic sequence and the cDNA sequence and also the description of methods for obtaining them are glyoxal oxidases from two different subdivisions of phytopathogenic fungi which are suitable for use in methods for identifying fungicides, it being possible to characterize and further develop, with the aid of the corresponding target, viz. glyoxal oxidase, these fungicides which have been identified.

The present invention therefore provides for the first time complete genomic sequences or the cDNA of glyoxal oxidases of pathogenic fungi and describes their use or the use of the polypeptide encoded by them for identifying inhibitors of the enzyme, and their use as fungicides.

The present invention therefore relates to nucleic acids which encode complete fungal glyoxal oxidases, with the exception of the Phanerochaete chrysosporium nucleic acid sequences encoding glyoxal oxidase (Kersten et al., 1995), PCGLX1G_{—}1 PRT with 559 amino acids (accessible at the EMBL under the Accession No. L47286 or at SPTREMBL under the Accession No. Q01772; (protein ID=AAA87594.1)), and PCGLX2G_{—}1 PRT with 559 amino acids (accessible at the EMBL under the Accession No. L47287 or at SPTREMBL under the Accession No. Q01773 (protein ID=AAA87595.1)). The protein sequences are identical with the exception of one amino acid substitution Lys 308 by Thr 308. The identity of the nucleotide sequences is 98%.

Using the nucleic acids according to the invention, it was likewise possible to identify further nucleic acid sequences from other fungi, which nucleic acid sequences enclode glyoxal oxidase, which, while having been available to the public as results in context with genome projects, have not had a function or biological importance assigned to them. These are sequences from Cryptococcus neoformans, a fungus which is pathogenic to humans held responsible for cryptococcal meningitis and pneumonia (see CRYNE_cneo 001022. contig 6786 (4064 bp), homology region: 2704-1393, CRYNE_cneo 001022.contig 7883 (13487 bp); homology regions: 916-1695, 468-2185, 2100-2345, CRYNE b6f10cnf1; homology region: 1-564, CRYNE_{—}4_contig 456; homology region: 930-19 and CRYNE_cneo001022. contig 6828 (4546 bp); homology region: 4364-3840), from the Ascomyceta Neurospora crassa, which is known as bread mould (see NEUCR_contig 1887 (supercontig 127); homology region: 14411-15889) and from the phytopathogenic rice blast fungus Magnaporthe grisea. It has thus been found that glyoxal oxidase also occurs in fungi which are pathogenic to humans. It can be assumed that in these fungi which are pathogenic to humans, too, the enzyme plays a not inconsiderable physiological role and is therefore an interesting target for enzyme modulators or plays a role as site of action for antimycotics in these fungi too.

In particular, the present invention relates to nucleic acids which encode glyoxal oxidases from phytopathogenic fungi, preferably from fungi of the subdivision Ascomycetes and Basidiomycetes, the genera Botrytis and Ustilago being especially preferred.

Very particularly preferably the present invention relates to nucleic acids which encode Ustilago maydis and Botrytis cinerea glyoxal oxidases.

The present invention particularly preferably relates to the nucleic acids encloding the Ustilago maydis glyoxal oxidases with the SEQ ED NO: 1, SEQ ID NO: 3, SEQ ID NO: 5 and SEQ ID NO: 7 and the nucleic acids encoding Botrytis cinerea glyoxal oxidases with the SEQ ID NO: 9 and SEQ ID NO: 11 and the nucleic acids encoding the polypeptides as shown in SEQ ID NO: 2, SEQ ID NO: 4, SEQ ID NO: 6, SEQ ID NO: 8, SEQ ID NO: 10 and SEQ ID NO: 12 or active fragments of these.

The nucleic acids according to the invention especially take the form of single-stranded or double-stranded deoxyribonucleic acids (DNA) or ribonucleic acids (RNA). Preferred embodiments are fragments of genomic DNA, which may contain introns, and cDNAs.

The nucleic acids according to the invention preferably take the form of DNA fragments which correspond to the cDNA of phytopathogenic fungi.

The nucleic acids according to the invention particularly preferably comprise a sequence selected from

a) a sequence as shown in SEQ ID NO: 1, SEQ ID NO: 3, SEQ ID NO: 5, SEQ ID NO: 7, SEQ ID NO: 9 and SEQ ID NO: 11,

b) sequences encoding a polypeptide which comprises the amino acid sequence as shown in SEQ ID NO: 2, SEQ ID NO: 4, SEQ ID NO: 6, SEQ ID NO: 8, SEQ ID NO: 10 or SEQ ID NO: 12,

c) sequences encoding a polypeptide which comprises the amino acids tyrosine 1 and 2, histidine 1 and 2 and cysteine according to formula (I), which are suitable for Cu²⁺ coordination,

d) part-sequences of the sequences defined under a) to c) which are at least 14 base pairs in length,

e) sequences with 50% identity, particularly preferably 70% identity, very particularly preferably 90% identity, with the sequences defined under a) to c),

f) sequences which are complementary to the sequences defined under a) to c), and

g) sequences which, owing to the degeneracy of the genetic code, encode the same amino acid sequence as the sequences defined under a) to c).

A very particularly preferred embodiment of the nucleic acids according to the invention is a cDNA molecule with the sequence as shown in SEQ ID NO: 1 and 3 or with the sequence SEQ ID NO: 5 or SEQ ID NO: 7 encoding an Ustilago maydis glyoxal oxidase.

A further very particularly preferred embodiment of the nucleic acids according to the invention is a cDNA molecule with the sequence as shown in SEQ ID NO: 9 or 11 encoding a Botrytis cinerea glyoxal oxidase.

The term “complete” glyoxal oxidase as used in the present context describes the glyoxal oxidases for which a complete coding region of a transcription unit starting with the ATG start codon and comprising all of the information-bearing exon regions of the gene present in the starting organisms and encoding glyoxal oxidases, and the signals required for correct transcriptional termination are present.

The term “active fragment” as used in the present context describes no longer complete nucleic acids encoding glyoxal oxidase which still encode polypeptides with the biological activity of a glyoxal oxidase, that is to say which are capable of catalysing the reaction

RCHO+O₂+H₂O.RCO₂H+H₂O

An activity assay can be used to determine whether this biological function does indeed still exist, which assay is based, for example, on detecting H₂O for example by acidification with H₂SO₄ and addition of TiOSO₄ solution (the formation of [TiO₂*aq]SO₄ leads to a yellowish-orange coloration). Glyoxal oxidase activity can also be observed in known glucose oxidases. In comparison with glyoxal oxidases, whose main activity is the catalysis of the above-shown reaction, however, this activity is markedly reduced. The term “biological activity” is therefore not intended to extend to those polypeptides such as glucose oxidase whose main activity is not the catalysis of this reaction. “Active fragments” are shorter than the above-described complete nucleic acids which encode glyoxal oxidase. In this context, nucleic acids may have been removed both at the 3′ and/or 5′ end(s) of the sequence; or else, parts of the sequence, which do not have a decisive adverse effect on the biological activity of glyoxal oxidase may have been deleted, i.e. removed. A lower or else, if appropriate, increased activity, which still allows the characterization or use of the resulting glyoxal oxidase fragments, is considered as sufficient for the purposes of the term as used herein. The term “active fragment” may also refer to the glyoxal oxidase amino acid sequence, in which case it applies, analogously, to what has been said above, to those polypeptides which in comparison with the above-defined complete sequence no longer contain certain portions, but where no decisive adverse effect on the biological activity of the enzyme has been exerted.

The preferred length of these fragments is 1200 nucleobases, preferably 900 nucleobases, very particularly preferably 300 nucleobases, or 400 amino acids, preferably 300 amino acids, very particularly preferably 100 amino acids.

The term “gene” as used in the present context is the name for a segment from the genome of a cell, which segment is responsible for synthesis of a polypeptide chain.

The term “to hybridize” as used in the present context describes the process in which a single-stranded nucleic acid molecule undergoes base pairing with a complementary strand. This is especially relevant for short regions spanning consensus sequences or other known regions of nucleic acids according to the invention, which regions are advantageously used for carrying out PCR experiments for identifying further nucleic acids encoding glyoxal oxidases. For example, starting from the sequence information disclosed herein, DNA fragments of further homologous genes or from fungi other than Ustilago maydis or Botrytis cinerea may be isolated in this manner, which DNA fragments encode glyoxal oxidases having the same properties as or similar properties to the glyoxal oxidases with the amino acid sequence as shown in SEQ ID NO: 1 and SEQ ID NO: 3, SEQ ID NO: 5, SEQ ID NO: 7, SEQ ID NO: 9 and SEQ ID NO: 11, respectively.

The term “cDNA” as used in the present context is the name for the single- or double-stranded copy of an RNA molecule and, being a copy of biologically active mRNA, is therefore free from introns, that is to say that all the coding regions of a gene are present in contiguous form.

Hybridization conditions as can be used mainly for the abovementioned PCR methods for identifying further fungal glyoxal oxidases are calculated approximatively using the following formula:

The melting point Tm=81.5° C.+16.6 log[c(Na⁺)]+0.41(% G+C))−500/n

(Lottspeich and Zorbas, 1998)

In this formula, c is the concentration and n the length of the hybridizing sequence segment in base pairs. For a sequence >100 bp, the term 500/n is dropped. Washing is effected with the highest stringency at a temperature of 5-15° C. under Tm and an ionic strength of 15 mM Na⁺ (corresponds to 0.1×SSC). If an RNA sample is used for hybridization, the melting point is 10-15° C. higher.

The degree of identity of the nucleic acids as described above is preferably determined with the aid of the program CLUSTALW or the program BLASTX Version 2.0.4 (Altschul et al., 1997).

The present invention furthermore relates to DNA constructs comprising a nucleic acid according to the invention and a homologous or heterologous promoter.

The term “homologous promoter” as used in the present context refers to a promoter which controls the expression of the gene in question in the source organism.

The term “heterologous promoter” as used in the present context refers to a promoter which has properties other than the promoter which controls the expression of the gene in question in the source organism.

The choice of heterologous promoters depends on whether pro- or eukaryotic cells or cell-free systems are used for expression. Examples of heterologous promoters are the cauliflower mosaic virus 35S promoter for plant cells, the alcohol dehydrogenase promoter for yeast cells, and the T3, T7 or SP6 promoters for prokaryotic cells or cell-free systems.

Fungal expression systems such as, for example, the Pichia pastoris system should preferably be used, transcription in this case being driven by the methanol-inducible AOX promoter. Heterologous expression for the Phanerochaete chrysosporium glyoxal oxidase has already been demonstrated for this system (Whittaker, M. et al., 1999).

The present invention furthermore relates to vectors containing a nucleic acid according to the invention, a regulatory region according to the invention or a DNA construct according to the invention. Vectors which can be used are all those phages, plasmids, phagemids, phasmids, cosmids, YACs, BACs, artificial chromosomes or particle bombardment particles which are used in molecular-biological laboratories.

Preferred vectors are pBIN (Bevan, 1984) and its derivatives for plant cells, pFL61 (Minet et al., 1992) or, for example, the p4XXprom. vector series (Mumberg et al., 1995) for yeast cells, pSPORT vectors (Life Technologies) for bacterial cells, or the Gateway vectors (Life Technologies) for a variety of expression systems in bacterial cells, plants, P. pastoris, S. cerevisiae or insect cells.

The present invention also relates to host cells containing a nucleic acid according to the invention, a DNA construct according to the invention or a vector according to the invention.

The term “host cell” as used in the present context refers to cells which do not naturally contain the nucleic acids according to the invention.

Suitable host cells are not only prokaryotic cells, preferably E. coli, but also eukaryotic cells such as cells of Saccharomyces cerevisiae, Pichia pastoris , insects, plants, frog oocytes and mammalian cell lines.

Fungal cells such as, for example, of Saccharomyces cerevisiae, Aspergillus nidulans and Pichia pastoris are preferably used for expression. Phanerochaete chrysosporium glyoxal oxidase was successfully expressed for example in A. nidulans and P. pastoris (Kersten et al., 1995; Whittaker et al., 1999).

Others which can be used for expressing the polypeptides according to the invention are, in particular, Ustilago maydis cells. Cells which are particularly suitable for this purpose are cells of a U. maydis strain which has been deposited at the Deutsche Sammlung von Mikroorganismen und Zellkulturen GmbH [German collection of microorganisms and cell cultures] (DSMZ), Mascheroder Weg 1 b in 38124 Brunswick on Sept. 13, 2001 under the number DSM 14 509.

These deposited cells were obtained as described in Example 3 and can be distinguished for example with the assay, shown in Example 4, of wild-type cells of the original strain. The strain with the deposit number DSM 14 509 is capable of expressing the U. maydis glyoxal oxidase according to the invention in sufficient amount and activity to detect a glyoxal oxidase activity and to enable the strain to be used in a process according to the invention.

The strain with the deposit number DSM 14 509 is subject-matter of the present invention.

The present invention furthermore relates to polypeptides with the biological activity of glyoxal oxidases which are encoded by the nucleic acids according to the invention.

The polypeptides according to the invention preferably comprise an amino sequence selected from among

a) the sequence as shown in SEQ ID NO: 2, SEQ ID NO: 4, SEQ ID NO: 6, SEQ ID NO: 8, SEQ ID NO: 10 and SEQ ID NO: 12,

b) sequences comprising the amino acids tyrosine 1, tyrosine 2, histidine 1, histidine 2 and cysteine as shown in formula (I) which are suitable for Cu²⁺ coordination,

c) part-sequences of the sequences defined under a) and b) at least 15 amino acids in length,

d) sequences with at least 20%, preferably 25%, particularly preferably 40%, very particularly preferably 60% and most preferably 75% identity with the sequences defined under a) and b), and

e) sequences with the same biological activity as the sequences defined under a) to d).

The term “polypeptides” as used herein refers both to short amino acid chains, which are usually referred to as peptides, oligopeptides or oligomers and to longer amino acid chains which are usually referred to as proteins. It encompasses amino acid chains which may be modified either by natural processes, such as post-translational processing, or by chemical methods which are state of the art. Such modifications may occur at various points and a plurality of times in a polypeptide, such as, for example, on the peptide backbone, on the amino acid side chain, on the amino terminus and/or on the carboxyl terminus. They comprise, for example, acetylations, acylations, ADP ribosylations, amidations, covalent linkages with flavins, haem portions, nucleotides or nuceotide derivatives, lipids or lipid derivatives or phosphatidylinositol, cyclizations, formation of disulphide bridges, demethylations, cystine formations, formylations, gamma-carboxylations, glycosylations, hydroxylations, iodinations, methylations, myristoylations, oxidations, proteolytic processings, phosphorylations, selenylations and tRNA-mediated additions of amino acids.

The peptides according to the invention may be in the form of “mature” proteins or in the form of parts of larger proteins, for example as fusion proteins. They may furthermore have secretion or leader sequences, prosequences, sequences which make simple purification possible, such as polyhistidine residues, or additional stabilizing epitopes.

The polypeptides according to the invention, in particular the polypeptides as shown in SEQ ID NO: 2, 4, 6, 8, 10 and 12, need not constitute complete fungal glyoxal oxidases, but may also only constitute fragments of these as long as they still have a biological activity of the complete fungal glyoxal oxidases. Polypeptides which exert the same type of biological activity as a glyoxal oxidase with an amino acid sequence as shown in SEQ ID NO: 2, 4, 6, 8 or SEQ ID NO: 10 and 12 are still considered as being according to the invention. In this context, the polypeptides according to the invention need not be deducible from Ustilago maydis or Botrytis cinerea glyoxal oxidases or from phytopathogenic fungi, but may, for example owing to the relationship between the glyoxal oxidases, be derived from various organisms such as fungi which are pathogenic for humans or else from plants (see also FIG. 8). Polypeptides which are considered according to the invention are, above all, also those polypeptides which correspond to glyoxal oxidases for example of the following fungi, or fragments of these, and which still have their biological activity:

Plasmodiophoromycetes, Oomycetes, Chytridiomycetes, Zygomycetes, Ascomycetes, Basidiomycetes and Deuteromycetes, for example.

Pythium species such as, for example, Pythium ultimum, Phytophthora species such as, for example, Phytophthora infestans, Pseudoperonospora species such as, for example, Pseudoperonospora humuli or Pseudoperonospora cubensis, Plasmopara species such as, for example, Plasmopara viticola, Bremia species such as, for example, Bremia lactucae, Peronospora species such as, for example, Peronospora pisi or P. brassicae, Erysiphe species such as, for example, Erysiphe graminis, Sphaerotheca species such as, for example, Sphaerotheca fuliginea, Podosphaera species such as, for example, Podosphaera leucotricha, Venturia species such as, for example, Venturia inaequalis, Pyrenophora species such as, for example, Pyrenophora teres or P. graminea (conidial form: Drechslera, syn: Helminthosporium), Cochliobolus species such as, for example, Cochliobolus sativus (conidial form: Drechslera, syn: Helminthosporium), Uromyces species such as, for example, Uromyces appendiculatus, Puccinia species such as, for example, Puccinia recondita, Sclerotinia species such as, for example, Sclerotinia sclerotiorum, Tilletia species such as, for example, Tilletia caries; Ustilago species such as, for example, Ustilago nuda or Ustilago avenae, Pellicularia species such as, for example, Pellicularia sasakii, Pyricularia species such as, for example, Pyricularia oryzae, Fusarium species such as, for example, Fusarium culmorum, Botrytis species, Septoria species such as, for example, Septoria nodorum, Leptosphaeria species such as, for example, Leptosphaeria nodorum, Cercospora species such as, for example, Cercospora canescens, Alternaria species such as, for example, Alternaria brassicae or Pseudocercosporella species such as, for example, Pseudocercosporella herpotri-choides.

Others which are of particular interest are, for example, Magnaporthe grisea, Cochliobulus heterostrophus, Nectria hematococcus and Phytophthora species.

As has already been discussed above, the polypeptides according to the invention may also be used as a site of action for antimycotics and thus for the control of fungi which are pathogenic for humans or animals. Of particular interest in this context are, for example, the following fungi which are pathogenic to humans and which may cause the symptoms stated hereinbelow:

Dermatophytes such as, for example, Trichophyton spec., Microsporum spec., Epiderinophyton floccosum or Keratomyces ajelloi, which cause, for example, Athlete's foot (tinea pedis),

Yeasts such as, for example, Candida albicans, which causes soor oesophagitis and dermatitis, Candida glabrata, Candida krusei or Cryptococcus neoformans, which may cause, for example, pulmonal cryptococcosis or else torulosis,

Moulds such as, for example, Aspergillus fumigatus, A. flavus, A. niger, which cause, for example, bronchopulmonary aspergillosis or fungal sepsis, Mucor spec., Absidia spec., or Rhizopus spec., which cause, for example, zygomycoses (intravasal mycoses), Rhinosporidium seeberi, which causes, for example, chronic granulomatous pharyngitis and tracheitis, Madurella mycetomatis, which causes, for example, subcutaneous mycetomas, Histoplasma capsulatum, which causes, for example, reticuloendothelial cytomycosis and Darling's disease, Coccidioides immitis, which causes, for example, pulmonary coccidioidomycosis and sepsis, Paracoccidioides brasiliensis, which causes, for example, South American blastomycosis, Blastomyces dermatitidis, which causes, for example, Gilchrist's disease and North American blastomycosis, Loboa loboi, which causes, for example, keloid blastomycosis and Lobo's disease, and Sporothrix schenckii, which causes, for example, sporotrichosis (granulomatous dermal mycosis).

The polypeptides according to the invention may, by comparison with the corresponding region of naturally occurring glyoxal oxidases, have deletions or amino acid substitutions as long as they exert at least one biological activity of the complete glyoxal oxidase. Conservative substitutions are preferred. Such conservative substitutions comprise variations where one amino acid is replaced by another amino acid from the following group:

1. small aliphatic residues which are nonpolar or of low polarity: Ala, Ser, Thr, Pro and Gly;

2. polar, negatively charged residues and their amides: Asp, Asn, Glu und Gln;

3. polar, positively charged residues: His, Arg und Lys;

4. large aliphatic, nonpolar residues: Met, Leu, Ile, Val and Cys; and

5. aromatic residues: Phe, Tyr und Trp.

The following list shows preferred conservative substitutions:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val, Met |
| Leu | Ile, Val, Met |
| Lys | Arg |
| Met | Leu, Ile |
| Phe | Met, Leu, Tyr, Ile, Trp |
| Pro | Gly |
| Ser | Thr |
| Thr | Ser |
| Trp | Tyr |
| Tyr | Trp, Phe |
| Val | Ile, Leu |

The present invention thus also relates to polypeptides which act like glyoxal oxidase in at least the biochemical reaction of the formation of hydroxide peroxide by reducing oxygen in the conversion of glyoxal or methylglyoxal or their derivatives and which comprise an amino acid sequence which has at least 20% identity, preferably 25% identity, particularly preferably 40% identity, very particularly preferably 60% identity, most preferably 75% identity and finally absolutely preferably 90% identity with the sequence as shown in SEQ ID NO: 2 and 4 or SEQ ID NO: 6 or 8 and SEQ ID NO: 10 or 12 over a length of 100 amino acids, preferably 250 amino acids and particularly preferably over its entire length.

The degree of identity of the amino acid sequences is preferably determined with the aid of the BLASTP+BEAUTY program (Altschul et al., 1997).

A particularly preferred embodiment of the polypeptides according to the invention are glyoxal oxidases with an amino acid sequence as shown in SEQ ID NO: 2, 4, 6 and 8 and SEQ ID NO: 10 and 12.

Particularly preferably, the present invention extends to those polypeptides according to the invention which comprise the abovementioned amino acids which are suitable for forming a Cu²⁺ coordination site:

Tyrosine ligand 1: (for example Tyr 178 (U. maydis) or Tyr 273 (B. cinerea)),

Tyrosine ligand 2: (for example Tyr 452 (U. maydis) or Tyr 499 (B. cinerea)),

Histidine ligand 1: (for example His 453 (U. maydis) or His 500 (B. cinerea)),

Histidine ligand 2: (for example His 555 (U. maydis) or His 597 (B. cinerea)), und

Cysteine residue: (for example Cys 105 (U. maydis) or Cys 209 (B. cinerea)).

The nucleic acids according to the invention can be prepared in the conventional manner. For example, the nucleic acid molecules can be prepared by complete chemical synthesis. It is also possible for short pieces of the nucleic acids according to the invention to be synthesized chemically and for such oligonucleotides to be radiolabelled or else labelled with a fluorescent dye. The labelled oligonucleotides can also be used to search cDNA libraries generated starting from fungal mRNA. Clones to which the labelled oligonucleotides hybridize are selected for isolating DNA fragments in question. After characterization of the DNA isolated, the nucleic acids according to the invention are obtained in a simple manner.

The nucleic acids according to the invention can also be generated by PCR methods using chemically synthetized oligonucleotides.

The term “oligonucleotide(s)” as used in the present context refers to DNA molecules which consist of 10 or more nucleotides, preferably 15 to 30 nucleotides. They are synthesized chemically and can be used as probes.

The skilled worker knows that the polypeptides of the present invention can be obtained in various ways, for example by chemical methods like the solid-phase method. The use of recombinant methods is recommended for obtaining larger protein quantities. Expression of a cloned glyoxal oxidase gene or fragments thereof can be effected in a series of suitable host cells which are known to the skilled worker. To this end, a glyoxal oxidase gene is introduced into a host cell with the aid of known methods.

Integration of the cloned glyoxal oxidase gene into the chromosome of the host cell is within the scope of the present invention. Preferably, the gene or fragments thereof is, or are, introduced into a plasmid, and the coding regions of the glyoxal oxidase gene or fragments thereof is, or are, linked operably to a constitutive or inducible promoter. The Pichia pastoris expression system from Invitrogen is an example of a particularly suitable expression system. Vectors which are suitable for this purpose are, for example, pPICZ and its derivatives. Expression can be induced here with the aid of the AOX promoter by adding methanol. Moreover, expression in the U. maydis system would also be suitable. Here, expression of the glyoxal oxidase genes or of fragments thereof would be effected for example by the inducible crg1 promoter or the constitutive otef promotor (Bottin et al., 1996, Spelling et al., 1994).

The basic steps for generating recombinant glyoxal oxidases are:

1 . Obtaining a natural, synthetic or semisynthetic DNA which encodes a glyoxal oxidase.

2. Introducing this DNA into an expression vector which is suitable for expressing glyoxal oxidases, either alone or as fusion protein.

3. Transformation of a suitable, preferably eukaryotic, host cell with this expression vector.

4. Growing this transformed host cell in a manner which is suitable for expressing glyoxal oxidases.

5. Harvesting the cells and, if appropriate, purification of the glyoxal oxidases by suitable known methods.

In this context, the coding region of the glyoxal oxidases can be expressed in E. coli using the customary methods. Suitable expression systems for E. coli are commercially available, for example the expression vectors of the pET series, for example pET3a, pET23a, pET28a with His-tag or pET32a with His-tag for simple purification and thioredoxine fusion for increasing the solubility of the expressed enzyme, and pGEX with glutathione synthetase fusion, and also the pSPORT vectors. The expression vectors are transformed into λDE3 lysogenic E. coli strains, for example BL21(DE3), HMS 174(DE3) or AD494(DE3). After the cells have started to grow under standard conditions which are familiar to the skilled worker, IPTG is used to induce expression. After the cells have been induced, they are incubated for 3 to 24 hours at temperatures of from 4 to 37° C.

The cells are disrupted by sonification in break buffer (10 to 200 mM sodium phosphate, 100 to 500 mM NaCl, pH 5 to 8). The expressed protein can be purified via chromatographic methods, in the case of protein expressed with His-tag by chromatography on an Ni-NTA column.

Expression of the protein in insect cell cultures (for example Sf9 cells) is another advantageous approach.

As an alternative, the proteins may also be expressed in plants. Thus, for example, at least 3 glyoxal oxidase homologues exist in Arabidopsis thaliana (see FIG. 8), which emphasizes the possibility of expression in plants.

The present invention also relates to methods for finding chemical compounds which bind to the polypeptides according to the invention and alter their properties. Thus, modulators which affect the activity of the enzyme constitute new fungicidal active compounds which are capable of controlling the pathogenicity of the fungi. Modulators may be agonists or antagonists, or activators or inhibitors. Of particular interest are, in the case of glyoxal oxidase, inhibitors of this enzyme which can prevent the pathogenicity of the fungi by inactivating the enzyme.

The present invention therefore also particularly relates to the use of fungal glyoxal oxidases as targets for fungicides and to their use in methods of finding modulators of these polypeptides. In such methods, glyoxal oxidases can be employed directly in a host cell, in extracts or in purified form, or be generated indirectly via expression of the DNA encoding them. The polypeptides according to the invention which have been described hereinabove (Glo 2 and Gio 3 as shown in SEQ ID NO: 6 and SEQ ID NO: 8) are likewise suitable for this application. Independently of their immediate importance for the pathogenicity of the fungus, they have sufficient homology with Glo1 to be used likewise in methods of identifying modulators of the enzyme which then become active as fungicide.

The present invention therefore also relates to the use of nucleic acids encoding glyoxal oxidases according to the invention, of DNA constructs containing them, of host cells containing them, or of antibodies binding to the glyoxal oxidases according to the invention in methods of finding glyoxal oxidase modulators.

The term “agonist” as used in the present context refers to a molecule which promotes or enhances the glyoxal oxidase activity.

The term “antagonist” as used in the present context refers to a molecule which slows down or prevents the glyoxal oxidase activity.

The term “modulator” as used in the present context constitutes the generic term for agonist or antagonist. Modulators may be small organochemical molecules, peptides or antibodies which bind to the polypeptides according to the invention. Modulators may furthermore be small organochemical molecules, peptides or antibodies which bind to a molecule which, in turn, binds to the polypeptides according to the invention and thus influences their biological activity. Modulators may be natural substrates and ligands or structural or functional mimetics thereof. The term “modulator”, however, does not encompass the natural substrates of glyoxal oxidase such as, for example, oxygen, glyoxal and methylglyoxal.

The modulators are preferably small organochemical compounds.

Binding of the modulators to the glyoxal oxidases according to the invention may alter the cellular processes in a manner which leads to apathogenicity or death of the fungus treated therewith.

The use of the nucleic acids or polypeptides according to the invention in a method according to the invention makes it possible to find compounds which bind to the polypeptides according to the invention. These can then be used as fungicides, for example in plants, or as antimycotic active compounds in humans and animals. For example, host cells which contain the nucleic acids according to the invention and which express the corresponding polypeptides, or the gene products themselves, are brought into contact with a compound or a mixture of compounds under conditions which permit the interaction of at least one compound with the host cells, the receptors or the individual polypeptides.

In particular, the present invention relates to a method which is suitable for identifying fungicidal active compounds which bind to fungal polypeptides with the biological activity of a glyoxal oxidase, preferably to glyoxal oxidases from phytopathogenic fungi, particularly preferably to Ustilago or Botrytis glyoxal oxidases, and very particularly preferably to U. maydis and B. cinerea glyoxal oxidases and polypeptides with are homologous thereto and which have the abovementioned consensus sequence. However, the methods can also be carried out with a polypeptide which is homologous to the glyoxal oxidases according to the invention and which is derived from a species other than those mentioned herein. Methods which use glyoxal oxidases other than the one in accordance with the invention are encompassed by the present invention.

A large number of assay systems for testing compounds and natural extracts are designed for high throughput numbers in order to maximize the number of test substances in a given period. Assay systems based on cell-free processes require purified or semipurified protein. They are suitable for an “initial” assay which aims mainly at detecting a potential effect of a substance on the target protein. However, assay systems based on intact cells which produce sufficient quantities of the polypeptide in question may also be used. In the present case, the enzyme activity can also successfully be measured with intact cells which overproduce glyoxal oxidase, for example Ustilago maydis cells, analogously to the activity assay as described in Example 4.

Effects such as cell toxicity are generally ignored in these in vitro systems. The assay systems check both inhibitory, or suppressive, effects of the substances and stimulatory effects. The efficacy of the substance can be checked by concentration-dependent test series. Controls without test substances can be used for assessing the effects.

In order to find modulators, a synthetic reaction mix (for example products of the in-vitro translation) or a cellular component such as an extract or any other preparation containing the polypeptide can be incubated together with a labelled substrate or a ligand of the polypeptides in the presence and absence of a candidate molecule, which may be an agonist or antagonist. The ability of the candidate molecule to increase or inhibit the activity of the polypeptides according to the invention can be seen from an increased or reduced binding of the labelled ligand or from an increased or reduced conversion of the labelled substrate. Molecules which bind well and lead to an increased activity of the polypeptides according to the invention are agonists. Molecules which bind well, but counteract the biological activity of the polypeptides according to the invention, are probably good antagonists.

Modulators of the polypeptide according to the invention can also be found via enzyme tests. The change in enzyme activity by suitable modulators can either be measured directly or indirectly in a linked enzyme assay. The measurement can be carried out for example via changes in the absorption caused by the decrease or * increase of an optically active compound. Thus, for example, the release or consumption of hydrogen peroxide can be detected by decoloration of a phenol red solution in the presence of horseradish peroxidase (see Example 4, 10 and 11).

A further possibility of identifying substances which modulate the activity of the polypeptides according to the invention is what is known as a “scintillation proximity assay” (SPA), see EP 015 473. This assay system exploits the interaction of a polypeptide (for example U. maydis oder B. cinerea glyoxal oxidase) with a radiolabelled ligand (for example a small organic molecule or a second radiolabelled protein molecule). The polypeptide is bound to microspheres or beads provided with scintillating molecules. As the radioactivity decreases, the scintillating substance in the microsphere is excited by the subatomic particles of the radioactive marker and a detectable photon is emitted. The assay conditions are optimized so that only those particles emitted from the ligand lead to a signal which is emitted by a ligand bound to the polypeptide according to the invention.

In one possible embodiment, the U. maydis glyoxal oxidase, for example, is bound to the beads, either together with, or without, interacting or binding test substances. Test substances which can be used are, inter alia, fragments of the polypeptide according to the invention. When a binding ligand binds to the immobilized glyoxal oxidase, this ligand should inhibit or nullify an existing interaction between the immobilized glyoxal oxidase and the labelled ligand in order to bind itself in the zone of the contact area. Once binding to the immobilized glyoxal oxidase has taken place, it can be detected with reference to a flash of light. Accordingly, an existing complex between an immobilized and a free, labelled ligand is destroyed by the binding of a test substance, which leads to a decline in the intensity of the flash of light detected. In this case, the assay system takes the form of a complementary inhibition system.

A further example of a method with the aid of which modulators of the polypeptides according to the invention can be found is a displacement assay, in which the polypeptides according to the invention and a potential modulator are combined, under conditions which are suitable for this purpose, with a molecule which is known to bind to the polypeptides according to the invention, such as a natural substrate or ligand, or a substrate or ligand mimetic.

The term “competitor” as used in the present context refers to the property of the compounds to compete with other, possibly yet to be identified, compounds for binding to glyoxal oxidase and to displace the latter, or to be displaced by the latter, from the enzyme.

The present invention thus also relates to modulators, preferably inhibitors of the enzymatic activity of the glyoxal oxidases according to the invention, which are found with the aid of one of the methods described herein for identifying modulators of the glyoxal oxidase protein or a polypeptide which is homologous thereto.

It has not been disclosed as yet that glyoxal oxidases from phytopathogenic fungi constitute a new target for fungicides and that compounds which can be employed as fungicides may be found and developed with the aid of these glyoxal oxidases. This possibility is described and exemplified for the first time in the present application. Furthermore provided are the glyoxal oxidases required therefor, and methods for obtaining them and for identifying inhibitors of the enzyme.

The invention therefore furthermore relates to the use of glyoxal oxidase modulators as fungicides.

Fungicidal active compounds which are found with the aid of the polypeptides according to the invention can also interact with glyoxal oxidases from fungal species which are pathogenic for humans; it is not always necessary for the interaction with the different glyoxal oxidases which occur in these fungi to be equally pronounced.

The present invention therefore also relates to the use of inhibitors of polypeptides with the function of a glyoxal oxidase for preparing compositions for the treatment of diseases caused by fungi which are pathogenic for humans or animals.

The terms “fungicide” or “fungicidal” as used in the present context also encompass the terms “an antimycotic” or “antimycotic” for the purposes of the invention. The present invention furthermore comprises methods of finding chemical compounds which modify the expression of the polypeptides according to the invention. Such “expression modulators”, too, may be new fungicidal active compounds. Expression modulators can be small organochemical molecules, peptides or antibodies which bind to the regulatory regions of the nucleic acids encoding the polypeptides according to the invention. Moreover, expression modulators may be small organochemical molecules, peptides or antibodies which bind to a molecule which, in turn, binds to regulatory regions of the nucleic acids encoding the polypeptides according to the invention, thus influencing their expression. Expression modulators may also be antisense molecules.

The present invention also relates to expression modulators of glyoxal oxidases which are found with the aid of an above-described method of identifying expression modulators of the glyoxal oxidase proteins or polypeptides homologous thereto.

The present invention also relates to the use of expression modulators of the nucleic acids according to the invention as fungicides.

The methods according to the invention include high-throughput screening (HTS). Both host cells and cell-free preparations containing the nucleic acids according to the invention and/or the polypeptides according to the invention may be used for this purpose.

The invention furthermore relates to antibodies which bind specifically to the polypeptides according to the invention or fragments of these. Such antibodies are raised in the customary manner. For example, said antibodies may be produced by injecting a substantially immunocompetent host with an amount of a polypeptide according to the invention or fragment thereof which is effective for antibody production, and subsequently obtaining this antibody. Furthermore, an immortalized cell line which produces monoclonal antibodies may be obtained in a manner known per se. The antibodies may be labelled with a detection reagent, if appropriate. Preferred examples of such a detection reagent are enzymes, radiolabelled elements, fluorescent chemicals or biotin. Instead of the complete antibody, fragments which have the desired specific binding properties may also be employed.

The nucleic acids according to the invention can likewise be used for generating transgenic organisms such as bacteria, plants or fungi, preferably for generating transgenic plants and fungi, particularly preferably for generating transgenic fungi. These can be employed for example in assay systems which are based on an expression, of the polypeptides according to the invention or their variants, which deviates from the wild-type. They furthermore include all transgenic plants or fungi in which the expression of the polypeptides according to the invention or variants of these is altered by modifying genes other than those described hereinabove or by modifying gene control sequences (for example promoters).

The transgenic organisms are also of interest for (over)producing the polypeptide according to the invention for commercial or industrial purposes; here, for example, fungi (for example yeast or Ustilago maydis) which show a higher degree of expression of the polypeptide according to the invention in comparison with their natural form are particularly suitable for use in methods (indeed also HTS methods) for identifying modulators of the polypeptide.

Also of particular interest in this context is the use of the transgenic fungi according to the invention in papermaking, where coupling with the known lignin peroxidases, i.e. the exploitation of fungi which express both enzymes with an activity which may be increased, or else in higher quantities, is of particular interest for the degradation of lignin.

Conversely, a use of the inhibitors of polypeptides with the biological function of a glyoxal oxidase, which inhibitors have been identified by the methods according to the invention, is also of interest for the protection of materials. Fungi are a major problem in particular in the conservation of timber. Since the glyoxal oxidases provide hydrogen peroxide for the lignin peroxidase, even the most inert timber constituents are degraded with their aid. As a consequence, however, the inhibition of glyoxal oxidase with inhibitors according to the invention also inhibits the lignin peroxidases, and the decomposition of timber in the internal and external sector can thus be reduced.

Moreover, the transgenic organisms according to the invention, that is to say fungi, but also, for example, algae or other microorganisms, for example bacteria, can be used for detoxifying media, for example in wastewater, polluted watercourses, water treatment plants and the like. In this context, the ability of the polypeptides according to the invention, and of the corresponding transgenic organisms, can be exploited to oxidize aldehydes as a function of the substrate spectrum and to convert them into less reactive and less environmentally damaging acids. Glyoxal oxidase itself, which can be obtained for example from transgenic overproducers, is, however, also of interest for detoxifying the human or animal body or blood by removing methylglyoxal (Thornalley, 1996; Thornalley et al., 2001). The ability of cells transformed with, for example, Glo1 to degrade a variety of undesired substances is demonstrated in Example 11 and FIG. 13.

The nucleic acids according to the invention can also be used for the generation of transgenic plants which are distinguished by increased resistance to pathogens or environmental stress. A number of crops such as, for example, sunflowers, canola, alfalfa, soya beans, peanut, maize, sorghum, wheat or rice, and a multiplicity of flowers, trees, vegetable crops or fruit crops such as, for example, grapevine, tomato, apple or strawberry, are sensitive to fungi such as, for example, Botrytis cinerea or other fungal species which are distinguished by expressing hydrogen peroxide, which represents a way for the fungus to gain access to the plant in question. The glyoxal oxidase according to the invention is such an enzyme which produces hydrogen peroxide. The infection of a plant by a pathogen triggers, in many plants, the activation of various defence mechanisms which may be accompanied by what is known as a hypersensitivy response (HR) and/or by destruction of the host tissue at the site of penetration of the pathogen. This may prevent the pathogen from spreading in the host. In some cases, the plant thus also develops a systemic resistance (systemic aquired resistance, SAR) to the infection of pathogens which are taxonomically far removed from the original infecting pathogen. One of the first responses to pathogen infection which can be observed is the increased accumulation of superoxide anions, that is to say O₂, and/or hydrogen peroxide, that is to say H₂O₂. The accumulation of H₂O₂ can trigger the increased resistance response in various ways: 1. via a direct antimicrobial action, 2. by providing H₂O₂ as substrate for peroxidases which contribute to the polymerization of lignin and thus help strengthening cell walls, 3. by acting, in a mechanism yet to be clarified, as signal for activating the expression of genes which play a role in the plant's defence against infection, for example, in the stimulation of salicylic acid accumulation. Salicylic acid, in turn, is considered an endogenous trigger for the expression of genes which encode several pathogenesis-related proteins (PRPs), for example glucanases or chitinases. Moreover, salicylic acid may also increase the oxidative burst and thus accelerate its own synthesis in a sort of feedback process. Furthermore, salicylic acid may play a role in hypersensitive cell death by acting as an inhibitor of catalase, an enzyme which degrades H₂O₂. Finally, H₂O₂ can also trigger the synthesis of additional compounds which are suitable for defence, for example of phytoalexins or low-molecular-weight antimicrobial compounds.

The glyoxal oxidases described in the present application are therefore suitable for conferring, to plants, a signficant resistance to attacks by pathogens. Owing to the glyoxal oxidase activity, the transgenic plants are capable of expressing PRP genes and of accumulating salicylic acid. The DNA constructs used for transforming the plants may contain for example a constitutive promoter and also the coding sequence linked operably thereto as well as a marker gene permitting selection of the transformants. Further elements which can be used are terminators, polyadenylation sequences and nucleic acid sequences encoding signal peptides which govern the localization within a plant cell or secretion of the protein from this cell.

A multiplicity of methods for the transformation of plants is already known (see also, for example, Miki et al. (1993), Gruber and Grosby (1993) and Bevan et al., 1983). The most developed vector system for generating transgenic plants is a plasmid from the bacterium Agrobacterium tumefaciens (Bevan, 1984). In nature, A. tumefaciens infects plants and generates tumours termed crown galls. These tumours are caused by the Ti plasmid (tumour-inducing) of A. tumefaciens. The Ti plasmid incorporates part of its DNA, termed T-DNA, into the chromosomal DNA of the host plant. A means of removing the tumour-inducing regions from the DNA of the plasmid, but retaining its property of introducing genetic material into the plants, has been developed. Then, a foreign gene, for example one of the nucleic acids according to the invention, can be incorporated into the Ti plasmid with the aid of customary recombinant DNA techniques. The recombinant plasmid is then retransformed into A. tumefaciens. The strain can then be used for infecting a plant cell culture. However, the plasmid can also be inserted directly into the plants. Regeneration of such cells into intact organisms gives rise to plants containing the foreign gene and also expressing it, i.e. producing the desired gene product.

While A. tumefaciens infects dicotyledonous plants with ease, it is of limited use as vector for the transformation of monocotyledonous plants, which include a large number of agriculturally important crop plants such as maize, wheat or rice, since it does not infect these plants readily. Other techniques, for example “DNA guns”, what is known as the particle gun method, are available for the transformation of such plants. In this method, minute titanium or gold microspheres are fired into recipient cells or tissue, either by means of a gas discharge or by a powder explosion. The microspheres are coated with DNA of the genes of interest, whereby the latter reach the cells and are gradually detached from the spheres and incorporated into the genome of the host cells.

Only a few of the cells which are exposed to the foreign hereditary material are capable of integrating it stably into the endogenous hereditary material. In a tissue which is used for gene transfer, the nontransgenic cells predominate. During the regeneration into the intact plant, it is therefore necessary to apply a selection which provides an advantage for the transgenic cells. In practice, marker genes which are transferred into the plant cells are used for this purpose. The products of these genes inactivate an inhibitor, for example an antibiotic or herbicide, and thus allow the transgenic cells to grow on the nutrient medium supplemented with the inhibitor.

In the case of the transformation with A. tumefaciens, protoplasts (isolated cells without cell wall which, in culture, take up foreign DNA in the presence of certain chemicals or else when using electroporation) may be used instead of leaf segments. They are kept in tissue culture until a new cell wall has formed (for example approximately 2 days in the case of tobacco). Then, agrobacteria are added, and the tissue culture is continued. A simple method for the transient transformation of protoplasts with a DNA construct is incubation in the presence of polyethylene glycol (PEG 4000).

DNA may also be introduced into cells by means of electroporation. This is a physical method for increasing the uptake of DNA into live cells. Electrical pulses temporarily increase the permeability of a biomembrane without destroying the membrane.

DNA may also be introduced by microinjection. DNA is injected into the vicinity of the nucleus of a cell with the aid of glass capillaries. However, this is difficult in the case of plant cells, which have a rigid cell wall and a large vacuole.

A further possibility is to exploit ultrasound: when cells are sonicated with soundwaves above the frequency range of hearing in humans (above 20 kHz), a temporary permeability of the membranes is also observed. When carrying out this method, the amplitude of the soundwaves must be adjusted very precisely since, otherwise, the sonicated cells burst and are destroyed.

Methods of generating transgenic plants according to the present invention or suitable constructs comprising, for example, signal sequences for governing expression or suitable promoters have been described, inter alia, for transgenic plants which express the above-described glucose oxidase (for example from A. niger) (CN 12 29 139, U.S. Pat. No. 5,516,671, WO 95/21924, WO 99/04012, WO 95/14784). Similar methods may also be used to obtain transgenic plants according to the invention.

A wide range of possibilities exists for the transformation of fungi. Besides protoplast transformation (see Example 2 and Schulz et al., 1990), further customary methods are available for this purpose. The lithium acetate method is frequently used for yeasts (Gietz et al., 1997). Here, the yeast cells are made competent for the uptake of DNA by chemical means. In the case of electroporation, the DNA which has been loaded is introduced into the cells by a pulse of current. Another method is the transformation by Agrobacterium tumefaciens. Starting from plasmids, this bacterium is capable of introducing DNA into foreign organisms. When heterologous sequences are introduced into this plasmid, the target cell is transformed.

The invention thus also relates to transgenic plants or fungi which contain at least one of the nucleic acids according to the invention, preferably transgenic plants such as Arabidopsis species or transgenic fungi such as yeast species or Ustilago species, and their transgenic progeny. They also encompass the plant parts, protoplasts, plant tissues or plant propagation materials of the transgenic plants, or the individual cells, fungal tissue, fruiting bodies, mycelia and spores of the transgenic fungi which contain the nucleic acids according to the invention. Preferably, the transgenic plants or fungi contain the polypeptides according to the invention in a form which deviates from the wild-type. However, those transgenic plants or fungi which are naturally characterized by only a very low degree of expression, or none at all, of the polypeptide according to the invention are also considered as being according to the invention.

Accordingly, the present invention likewise relates to transgenic plants and fungi in which modifications in the sequence encoding polypeptides with the activity of a glyoxal oxidase have been generated and which have then been selected for the suitability for generating a polypeptide according to the invention and/or an increase or reduction, obtained by mutagenesis, in the biological activity or the amount of the polypeptide according to the invention which is present in the plants or fungi.

The term “mutagenesis” as used in the present context refers to a method of increasing the spontaneous mutation rate and thus of isolating mutants. In this context, mutants can be generated in vivo with the aid of mutagens, for example with chemical compounds or physical factors which are suitable for triggering mutations (for example base analogues, UV rays and the like). The desired mutants can be obtained by selecting towards a particular phenotype. The position of the mutations on the chromosomes can be determined in relation to other, known mutations by recombination analyses. The gene in question can be identified by complementation experiments using a gene library. Mutations can also be introduced into chromosomal or extrachromosomal DNA in a directed fashion (in-vitro mutagenesis, site-directed mutagenesis, error-prone PCR and the like).

The term “mutant” as used in the present context refers to an organism which bears a modified (mutated) gene. A mutant is defined by comparison with the wild-type which bears the unmodified gene.

The term “resistance” as used in the present context refers to forms of “resisting ability” based on a wide range of mechanisms. Forms of “active resistance” are “immunity” (=resistance of unsusceptible plants) and “tolerance” (=resistance of the plants which are susceptible to the pathogen). An intermediate form is “translocation resistance”, where the pathogen remains locally in individual cells, cell complexes or plant organs. There are transitional forms between the three types of resistance.

The term “pathogen” or “attack by pathogens” as used in the present context refers to organisms, in particular fungi, which are capable of attacking and damaging or destroying a plant. The damage can be based on a wide range of symptoms, such as, for example, discolorations, necroses, growth inhibition or the dying-off of parts of the plant. Organisms, which reduce the value of a plant by bringing about certain symptoms (for example discolorations, necroses), but do not lead to a plant or plant part dying off, are also termed pathogens.

Besides the generation of transgenic plants, another route which is based on the present invention may be taken to increase the resistance of plants to attack by pathogens.

Thus, it has been found that mutants of, for example, Botrytis cinerea in which the glyoxal oxidase encoding gene (cf. SEQ ID NO: 9 and 11) has been inactivated or deleted (cf. Example 9, generation of B. cinerea BcGlyox1 knock-out mutants) are no longer capable of causing the symptoms of damage, in plants, which are typical for this fungus (cf. Example 9 and FIG. 9 to 12). In plants which have been inoculated with conidia of this mutant, the mutants triggered a response as described above to the presence of the fungus, which response led to the establishment of local and systemic resistance. The establishment of resistance can be tested readily by bringing an untreated plant and a plant which has been treated with a fungus no longer capable of expression glyoxal oxidase into contact with a pathogen (cf. Example 9) and observing the damage of the plant over a specific period. The acquired resistance of the plant is unspecific in this context, that is to say it is directed not only against the fungus used for inducing or increasing the resistance, but induces a defence mechanism directed against attack by a wide range of pathogens.

The present invention therefore also relates to a method of inducing or increasing the resistance of a plant to attack by pathogens, by bringing a plant into contact with a fungus which is no longer capable of expresssing glyoxal oxidase and whose wild-type is preferably counted amongst the phytopathogenic fungi. These fungi are preferably fungi in which the gene(s) encoding glyoxal oxidase has, or have, been inactivated or deleted. Methods of deleting or inactivating a gene are known to the skilled worker (cf. also Example 9). Knock-out mutants of the fungus in question are preferably used. In addition to the abovementioned fungus Botrytis cinerea or its mutants, other fungi with a suitable deletion or inactivation of the glyoxal oxidase gene are also suitable for the treatment of plants, for example U. maydis mutants.

The present invention therefore also relates to the use of fungi, preferably phytopathogenic fungi, which are no longer capable of expressing glyoxal oxidase as plant treatment agents for increasing or inducing a resistance of the treated plant to attack by pathogens. The B. cinerea BcGlyox1 mutant according to the invention is particularly preferably used for this purpose.

The examples which follow now demonstrate that, surprisingly, the polypeptides according to the invention constitute an enzyme which is essential for pathogenicity in fungi and furthermore demonstrate that the enzyme is a suitable target protein for identifying fungicides, that it can be used in methods for identifying fungicidally active compounds and that the glyoxal oxidase modulators identified in the corresponding methods can be used as fungicides.

Moreover, an example of a method of measuring the enzymatic activity of glyoxal oxidases which can be used in methods for identifying modulators of the enzyme is described (Example 10 and 22), the methods according to the invention for identifying fungicides not being limited to the method stated.

Likewise, the examples which follow are not limited to Ustilago maydis or Botrytis cinerea. Analogous methods and results are also obtained in connection with other fungi.

### EXAMPLES

### Example 1

### Isolation of the Nucleic Acid Encoding the U. maydis Glyoxal Oxidase (“Plasmid Rescue”)

The plasmid rescue was carried out as described by Bölker et al., 1995. The genomic U. maydis DNA was cut with MulI, religated and transformed into E. coli strain DH5α by electroporation.

### U. maydis Culture

The strains were grown at 28° C. in PD medium or YEPS medium (Tsukada et al., 1988). After strains had been applied in the form of drops to PD plate media containing 1% charcoal, the development of dikaryotic filaments was observed (Holliday, 1974). Pathogenicity tests were carried out as described (Gillessen et al., 1992). Overnight cultures of the strains were resuspended at a concentration of 4×10⁷ cells and injected into young maize plants (Gaspar Flint). At least 80 plants were infected for each strain or each strain combination and examined for anthocyanin development and tumour development after 7 to 21 days.

### Imaging

The morphology of individual Ustilago maydis cells was analysed using a Zeiss axioscope and what is known as the differential interference contrast method. Micrographs of the cells were taken (Kodak T-64, magnification factor 1000).

### Example 2

### Generation of glo1 and glo2 Knock-out Mutants in U. maydis

### Generation of the Knock-out Cassette

Molecular-biological standard methods were carried out as described by Sambrook et al., 1989. To generate glo1 zero mutants, the 5′ and 3′ flanks of the glo1 gene were amplified by PCR. Genomic DNA of the strain UM518 was used as template. The primers LB2 with the sequence 5′-cacggcctgagtggccggtgtgtaaacgatcctttctggaag-3′ and LB1 with the sequence 5′-cctccaagtttcgagatatcgacc-3′ were employed for the 5′ flank (1151 bp). The primers RB1 (5′-gtgggccatctaggccgtcaacagcaccaaattcacagcc-3′) and RB2 (5′-atcgtagctcgagtgtatgcttcc-3′) were used for the 3′ flank (1249 bp). The cleavage sites Sfi I (a) and Sfi I (b) were introduced with the primers LB2 and RB1. The amplicons were restricted with Sfi I and ligated with the 1884 bp Sfi I fragment, which had been isolated from the vector pBS (hygromycinB cassette). The 4300 bp glo1 knock-out casette was amplified by PCR with the primers LB1 and RB2 (Kämper and Schreier, 2001).

### Preparation of U. maydis Protoplasts

50 ml of a culture in YEPS medium were grown at 28° C. to a cell density of approx. 5×10⁷/ml (OD 0.6 to 1.0) and then spun down for 7 minutes at 2500 g (Hereaus, 3500 rpm) in 50 ml Falcon tubes. The cell pellet was resuspended in 25 ml of SCS buffer (20 mM sodium citrate pH 5.8, 1.0 M sorbitol, (mix 20 mM sodium citrate/1.0 M sorbitol and 20 mM citric acid/1.0 M sorbitol and bring to pH 5.8 using pH meter)), spun again for 7 minutes at 2500 g (3500 rpm), and the pellet was resuspended in 2 ml of SCS buffer, pH 5.8, supplemented with 2.5 mg/ml Novozym 234. Protoplasts were released at room temperature, and the process was monitored under the microscope every 5 minutes. The protoplasts were then mixed with 10 ml of SCS buffer and spun for 10 minutes at 1100 g (2300 rpm), and the supernatant was discarded. The pellet was carefully resuspended in 10 ml of SCS buffer and spun again. The washing process with SCS buffer was repeated twice, and the pellet was washed in 10 ml of STC buffer. Finally, the pellet was resuspended in 500 μl of cold STC buffer (10 mM Tris/HCl pH 7.5, 1.0 M sorbitol, 100 mM CaCl2) and kept on ice. Aliquots can be stored for several months at −80° C.

### Transformation of U. maydis

U. maydis was transformed by the method of Schulz et al., 1990. Genomic U. maydis DNA was isolated as described by Hoffmann and Winston 1987.

To this end, a maximum of 10 μl of DNA (optimally 3-5 μg) were transferred into a 2 ml Eppendorf tube, 1 μl of heparin (15 μg/μl) (SIGMA H3125) was added, and 50 μl of protoplasts were then added and incubed on ice for 10 minutes. 500 μl of 40% (w/w) PEG3350 (SIGMA P3640) in STC (filter-sterilized) were added and mixed carefully with the protoplast suspension, and the mixture was incubated on ice for 15 minutes. The mixture was plated onto gradient plates (bottom agar: 10 ml YEPS-1.5% agar-IM sorbitol supplemented with antibiotic; shortly before plating, the bottom agar layer was covered with 10 ml YEPS-1.5% agar-IM sorbitol, the protoplasts were plated and the plates were incubated for 3-4 days at 28° C.).

For the Southern analysis, the DNA was restricted with EcoRI and XhoI. Detection was performed with a 1249 bp PCR fragment (RB1/RB2) labelled with digoxigenin (Roche) as DNA probe.

### Example 3

### Overproduction of Glo1

For the overproduction of Glo1, a 3400 bp fragment, which contained the glo1 gene, was amplified with the primers 5′glo1 (5′-cccgggatacgaggcacctctcctcatc-3′) and 3′glo1Not (5′-gcggccgcgaattggtcagacgaatccg-3′). The amplicon was cloned into the vector pCR-Topo2.1 (Invitrogen). The glo1 fragment was reisolated by restriction with SmaI and NotI and cloned into the respective cleavage sites of pCA123. pCA123 is a plasmid obtained from the plasmid potef-SG (Spellig et al., 1996), where the otef promoter was isolated from potef-SG as an 89u0 bp PvuII/NcoI fragment and ligated into the PvuII/NcoI-cut vector pTEF-SG (Spellig et al., 1996). In the resulting plasmid, the SGFP gene was excised by restriction with NcoI/NotI and replaced by the NcoI/NotI-cut EGFP allele from pEGFP-N1 (Clontech). The resulting plasmid is named pCA123. The plasmid pCA929, which finally resulted from pCA 123, was linearized with SspI and transformed into U. maydis. The U. maydis strain used is accessible in the public collection of the Deutsche Sammlung von Mikroorganismen und Zellkulturen [German collection of microorganisms and cell cultures] in Brunswick under the strain number UM 521. The transformands were transformed with the construct glo1-1 and selected for cbx resistance (Keon et al., 1991).

The resulting strain Ustilago maydis BAY-CA95 can be used for overproducing the polypeptide Glo1 according to the invention. It was deposited at the DSMZ in Brunswick under the number DSM 14 509.

### Example 4

### Cell Disruption, Fractionation of the Extract, and Assaying the Enzyme Activity

The glyoxal oxidase activity was determined in intact cells, in cell extracts and in membrane fractions.

Cells of the Ustilago maydis strain deposited under the deposit number DSM 14 509 which express glyoxal oxidase were grown in minimal medium or PD medium to an OD_{600 nm} of 0.6 to 3, spun down and brought to an OD_{600 nm} of 20 by resuspending. Cell extracts were obtained by comminuting in liquid nitrogen in a pestle and mortar. All the following steps were carried out at 4° C. Cell residues and cell debris were removed by fractional centrifugation at 5000 rpm and 8000 rpm. Membranes were isolated by spinning for 45 minutes at 13 000 rpm. The membrane sediment was resuspended in 50 mM Tris/HCl buffer pH 8 supplemented with 0.5% Tween-20.

The Glo1 activity can be measured by coupling the enzymatic reaction with phenol red and peroxidase. The glyoxal oxidase activity was detected by coupling with a horseradish peroxidase (HRP) reaction with phenol red as substrate. Here, the assay volume of 50 μl consists of 10 μl of sample, 15 μl of 50 mM potassium phosphate buffer pH 6, 5 μl of a 100 mM methylglyoxal solution, 5 μl of HRP (190 U/ml) and 5 μl of a 56 mM phenol red solution (Kersten and Kirk 1987). After incubation for 4 hours at 28° C., NaOH was added up to a concentration of 0.5 M. The absorption A_{550 nm} was determined in a “Tecan plus” reader. Active enzyme is identified with reference to the decoloration of the phenol red.

Substances or substance mixtures which influence the activity of the enzyme can be identified by comparing the enzyme activity in the presence and absence of this test substance using suitable controls in the experiment.

Other substrates for glyoxal oxidase may also be used in the above-described process, in which methylglyoxal was used as substrate. Besides intact cells, in turn, membrane fractions may be employed. The utilizable substrates also include, for example, formaldehyde, acetaldehyde, glycolaldehyde, glyoxal, glyoxalate, glycerol aldehyde, dihydroxyacetone, hydroxyacetone and glutaraldehyde, but the amount of the H₂O₂ formed does not necessarily have to be the same under otherwise identical conditions.

### Example 5

### Isolation of the Nucleic Acid Encoding B. cinerea Glyoxal Oxidase

### Strains Used

The wild-type strain B05.10 was used for analysis, transformation and as wild-type comparison strain. B05.10 is a derivative of the strain SAS56 (van der Vlugt-Bergmans et al, 1993).

### Culture on Agar Plates

B. cinerea was grown at 20° C. in the dark on plates containing Oxoid malt agar or Oxoid Czapek-Dox agar (Sucrose 30.00 g, NaNO₃ 3.00 g, MgSO₄×7 H₂O 0.50 g, KCl 0.50 g, FeSO₄×7 H₂O 0.01 g, K₂HPO₄ 1.00 g, agar 13.00 g, distilled H₂O 1000.00 ml; bring pH to 7.2), supplemented with various carbon sources.

### Isolation of the Conidia

Conidia (asexual spores of higher fungi) isolation was done using plates which had been covered completely by mycelial growth. To induce sporulation on these plates, they were exposed to UV light (270 nm-370 nm) for 16 hours. The conidia were washed off from plates on which the fungi sporulated 7 to 14 days post-induction using 5 ml of sterile water containing 0.05% (v/v) Tween 80. The suspension was filtered through glass wool, washed once by centrifugation (5′) at 114×g and resuspended in sterile water.

### Storage of B. cinerea Strains and of Knock-out Mutants

Conidia of the wild-type and of the mutants of B. cinerea were frozen at −80° C. in 75% (v/v) glycerol containing 12 mM NaCl.

### Isolation of the Glyoxal Oxidase Gene bcglvox1

A genomic library of B. cinerea, strain SAS56, in lambda EMBL3 (van der Vlugt-Bergmans et al., 1997) was screened for the presence of a glyoxal oxidase gene. The probe used was a cDNA fragment of strain T4 which was 385 base pairs in length and which had been identified as possibly homologous with the Phanerochaete chrysosporium glyoxal oxidase. The fragment is deposited in the EMBL database under the accession No. AL113811. Various hybridizing phages were purified, and the phage DNA was isolated. A hybridizing 4.1 kbp BamHI restriction fragment from one of the phages was cloned into a pBluescript®SKII(−) phagemid from Stratagene and subsequently sequenced. The characteristics of the cloned fragment are shown in FIG. 5.

### Example 6

### Southern Blot Analysis of the Genomic DNA

### Isolation of the Genomic DNA

The mycelium of a liquid culture was harvested by filtration through Miracloth (Calbiochem) and freeze-dried. The dried mycelium was homogenized in liquid nitrogen. 3 ml TES (100 mM Tris-HCl pH 8.0, 10 mM EDTA and 2% (w/v) SDS) and 60 μl proteinase K (20 μg/μl) were added, and the suspension was incubated for one hour at 60° C. 840 μl of 5M NaCl and 130 μl of 10% (w/v) N-cetyl-N,N,N-trimethylammonium bromide (CTAB) were subsequently added and the incubation was continued for 20 minutes at 65° C. The suspension was then processed by adding 4.2 ml of chloroform/isoamyl alcohol (24:1), followed by briefly mixing and 30 minutes incubation on ice and subsequent spinning for 5 minutes at 18 000×g. The aqueous upper phase was removed and 1350 μl of 7.5 M NH₄ acetate were added, and the mixture was incubated on ice for one hour and spun for 15 minutes at 18 000×g. 0.7 volume of isopropanol was added to precipitate the DNA. The DNA was removed by means of a glass rod, washed in 70% (v/v) ethanol and dried. The genomic DNA was finally dissolved in 1 ml of TE (10 mM Tris-HCl pH 7.5 and 0.1 mM EDTA, 2.5 U RNase A), incubated for 30 minutes at 50° C. and precipitated with ethanol.

### Southern Blot Analysis

1 μg of genomic DNA in a total volume of 100 μl was cleaved completely with the desired restriction enzyme. DNA fragments were separated on a 0.8% (w/v) agarose gel and subsequently blotted on Hybond™-N⁺ membranes from Amersham as specified in the protocol for an alkaline blot. To this end, the DNA-containing gel was first placed into 0.25 M HCl until the dyes had changed color. After washing the gel in distilled water, a capillary blot was carried out as described by Sambrook et al. (1989), using 0.4 M NaOH as blotting solution. After transfer of the DNA, the membrane was washed briefly in 2×SSC (0.3 M NaCl and 0.03 M sodium citrate, pH 7) and dried. The DNA was immobilized on the membrane by UV treatment (312 nm, 0.6 J/cm²).

Radiolabelled probes were prepared with the aid of the “Random Primers DNA Labeling System” (Life Technologies). To this end, 20 ng of the DNA fragments (“probe”, see FIG. 5) were labelled in accordance with the manufacturer's protocol. The labelled DNA fragments were purified over a Sephadex G50 column.

Hybridization was performed as described by Church and Gilbert (1984). To this end, the blot was prehybridized for 30 minutes at 65° C. in hybridization buffer (0.25 M phosphate buffer, pH 7.2, 1 mM EDTA, 1% (w/v) BSA and 7% (w/v) SDS). The blot was then hybridized for 40 hours at 65° C. with hybridization buffer containing the labelled probe. The blots were washed three times (30 minutes, 65° C. in 2×SSC and 0.1% (w/v) SDS). Autoradiography was carried out using a Kodak X-OMAT AR film.

The hybridization results are shown in FIG. 6. Single bands were identified with the probe in all three restrictions (SalI, BamHI and EcoRI). The BamHI fragment which hybridized was 4 kbp in size.

### Example 7

### Cloning the cDNA

Complete cDNA fragments were obtained by means of the Superscript™ One-Step RT-PCR system from Life Technologies. To this end, 0.1 μg of the total RNA which had been isolated from aus B. cinerea, strain B05.10, following the TRIzol protocol using the TRIzol® reagent (TRIzol reagents are monophasic solutions of phenol and guanidinium thiocyanate; after the addition of chloroform and subsequent centrifugation, the RNA is precipitated from the aqueous phase using isopropanol), subjected to reverse transcription and amplified with the aid of gene-specific primers. The cDNA was cloned directly into the vector pCR® 4-TOPO® (Invitrogen) and sequenced completely.

The cDNA sequence confirms the existence of an intron between the sequences which encode the chitin-binding domain and the glyoxal oxidase domain.

### Example 8

### Expression of BcGlyox1

The expression of BcGlyox1 was studied with reference to the course of the infection over time of tomato leaves. The conidia of the B. cinerea strain B05.10 were preincubated for 2 hours in B5 medium supplemented with 10 mM glucose and 10 mM (NH₄)H₂PO₄ to stimulate germination. The leaves of tomatoes (Lycopersicon esculentum cultivar moneymaker genotype Cf4) were inoculated by spraying with the medium, with contained 10⁶ spores per ml. The leaves were incubated at 20° C. and an atmospheric humidity of >95% and subsequently harvested at regular intervals post-inoculation and stored at −80° C.

The RNA was extracted from the mycelium which had been freeze-dried and homogenized in liquid nitrogen by comminuting the tissue into a powder using a pestle and mortar. 2 ml of guanidinium buffer pH 7.0 were added per gram of material. The buffer was composed of 8.0 M guanidinium hydrochloride, 20 mM 2-[N-morpholino]ethanesulphonic acid (MES), 20 mM EDTA and 50 mM β-mercaptoethanol, pH 7.0. The suspension was extracted twice, once with an equal volume of phenol/chloroform/isoamyl alcohol (IAA) (25:24:1 v/v/v) and once with chloroform/IAA (24:1 v/v). After centrifugation for 45 minutes at 12 000×g at 4° C., a third of the volume of 8 M LiCl was added to the aqueous phase. The suspension was subsequently incubated overnight on ice and spun for 15 minutes at 12 000×g. The precipitate was washed once with 2 M LiCl and twice with 70% (v/v) ethanol, dried in the air and dissolved in 1 ml of TE. The RNA concentration was determined spectrophotometrically at 260 nm. As an alternative, the TRIzol® reagent (Life Technologies) was also used, in accordance with the manufacturer's instructions, to obtain the RNA from the freeze-dried material.

For running the total RNA in a gel electrophoresis, the samples were denatured as follows. 3.6 μl of 6 M deionized glyoxal, 10.7 μl of dimethyl sulphoxide and 2.0 μl of 0.1 M sodium phosphate buffer pH 7 were added to 10 μg of the total RNA in 3.7 μl of solution. The sample was incubated for 60 minutes at 50° C., spun briefly, frozen in liquid nitrogen and defrosted again on ice. The sample was separated in a 1.4% (w/v) agarose gel. Gel and running buffer contained 0.01 M sodium phosphate buffer pH 7.0. After the gel had been run, the separated RNA fragments were transferred to a Hybond™-N⁺ membrane (Amersham) by capillary blotting (Sambrook et al., 1989), using a blotting solution with 0.025 M sodium phosphate buffer, pH 7. After the RNA had been transferred, the membrane was dried and the RNA was immobilized on the membrane by UV treatment (312 nm, 0.6 J/cm²). The hybridization protocol is as stated for the DNA hybridization.

### Example 9

### Generation of B. cinerea BcGlyox1 Knock-out Mutants

### Vector Construction

B. cinerea was transformed with a vector for homologous recombination which contained the BCGlyox1 gene in which an NruI-HindIII fragment had been deleted and replaced by a hygromycin resistance cassette (pHyGLYOX1, see FIG. 8).

### Preparation of Protoplasts

To obtain protoplasts for transformation, 1 litre of 1% (w/v) malt extract (Oxoid) was inoculated with 2×10⁸ B. cinerea conidia (strain B05.10). After 2 hours, the germinating conidia were incubated for 24 hours at 20° C. in a rotary shaker at 180 rpm. The mycelium was harvested by means of a 22.4 μm screen and incubated in 50 ml of KC solution containing 0.6 M KCl and 50 mM CaCl₂, supplemented with 5 mg/ml Glucanex (thermostable beta-glucanase for hydrolysing beta-glucan polysaccharides). After the protoplasts had been prepared in this way, the suspension was filtered through a 22.4 μm and a 10 μm screen. The protoplasts were washed and resuspended to a concentration of 10⁷ protoplasts per 100 μl.

### Transformation and Selection of Transformants

2 μg of the transformation vector pHyGLYOX1 which had been cleaved with EcoRI and extracted with phenol were diluted in 95 μl of KC, and 2 μl of 5 mM spermidin were added. Following incubation on ice for 5 minutes, 100 μl of the protoplast suspension were added to the DNA, and everything was incubated on ice for a further 5 minutes. 100 μl of polyethylene glycol (PEG) solution containing 25% (v/v) PEG 3350 in 10 mM Tris-HCl, pH 7.4 and 50 mM CaCl₂ were added, and the suspension was mixed. After 20 minutes at room temperature, 500 μl of PEG were added, and the vessels were left to stand at room temperature for a further 10 minutes. Finally, 200 μl of KC solution were added.

The transformation reaction with the transformed protoplasts was mixed with 200 ml of SH agar and immediately distributed between 20 Petri dishes. SH agar contains 0.6 M sucrose, 5 mM HEPES pH 6.5, 1.2% (w/v) purified agar and 1 mM NH₄(H₂PO₄). After incubation at 20° C. for 24 hours, an equal volume of SH agar containing 50 μg/ml hygromycin was added. Individual colonies which appeared were transferred to malt agar plates containing 100 μg/ml hygromycin for further selection. Growing colonies were then transferred to malt agar plates which did not contain hygromycin, and sporulation was triggered by treatment with UV light (near UV). To obtain monospore isolates, the conidia were isolated, diluted and plated onto malt agar plates supplemented with 100 μg/ml hygromycin. The colonies obtained from these plates were isolated and used for further analysis.

### Southern Analysis of the Transformants

Transformants were subjected to Southern analysis. The DNA was isolated and cut with EcoRV, separated electrophoretically, blotted and hybridized with a probe (see above). In the case of knock-out transformants, such a hybridization should yield a 300 bp fragment. All transformants with a slow growth phenotype showed the 300 bp fragment.

### Growth Analysis of the Transformants

All of the transformants which had grown on plates with a high hygromycin content also grew normally on malt agar plates without hygromycin. When the transformants were grown on synthetic agar media which contained simple sugars as carbon source, the transformants grew slowly or ceased growing. Examples of the sugars tested were hexoses, pentoses and trioses. Both germination and hyphal development were adversely affected or prevented completely. The growth defect can be compensated for by addition of, for example, tryptone or peptone. The growth inhibition can be remedied completely by adding arginine to the medium. Concentrations of 100 μM arginine and higher are capable of completely restoring the growth of the fungus on media containing simple sugars.

### Bioassays

A bioassay was carried out to compare the virulence of BcGlyox1 mutants with that of the wild-type B. cinerea (strain B05.10).

Excised leaves and fruits of tomatoes (Lycopersicon esculentum) and apples (Alkmene and Cox Orange) were inoculated with a conidial suspension (Benito et al., 1998; ten Have et al., 1998). The excised flowers of roses and gerbera hybrids were dusted with dry conidia (van Kan et al., 1997). The inoculated host tissue was incubated at 15° C. in the dark (tomato leaves and fruits, roses and gerbera) or at 20° C. and in the light (apples).

The BcGlyox1 mutants tested were incapable of causing primary necrotic lesions in all of the experimental set-ups, while the wild-type caused primary lesions which in some cases spread to the neighbouring tissue (see FIGS. 9 to 12).

Since, unlike the wild-type, the BcGlyox1 mutants do not germinate in B5 medium in the presence of simple sugars (standard medium), germination was stimulated by preincubating the conidia for 2 hours at room temperature in a 1% strength malt extract. This led to efficient germination of wild-type and mutant. These preincubated suspensions were likewise used for inoculation to exclude virulence of the mutant owing to other defects or deficiencies. However, even these experiments demonstrated that the mutants are not capable of infecting the test tissue (FIGS. 9 to 12).

Finally, arginine was additionally added to the inoculation suspension in order to do away with the mutants' problems with the utilization of simple sugars. The inoculation of wounded apples with arginine-containing suspensions of conidia of the mutant and of the wild-type revealed that necrotic tissue developed in both cases. The lesions of the wild-type spread for a few days until, finally, all of the tissue had rotted. The lesions caused by the mutant spread for 2 to 3 days, whereupon spreading stopped completely.

### Example 10

### Detection of the Expression of Enzymatic Activity of Glyoxal Oxidase

The activity of glyoxal oxidase in vitro and in vivo, for example in the U. maydis cells according to the invention produced as described in Example 3 (CA95) can be detected on the basis of the conversion of the substrate methylglyoxal, exploiting the following reaction:

Step 1:

Methylglyoxal+O₂→pyruvate+H₂O₂

Step 2:

H₂O₂+10-acetyl-3,7-dihydroxyphenoxazine (Amplex Red®)→resorufin+H₂O

Amplex Red® reacts with H₂O₂ in a 1:1 stoichiometry, giving rise to resorufin (7-hydroxy-3H-phenoxazin-3-one sodium salt). The fluorescence is measured at an excitation wavelength of 550 nm and an emission of 595 nm. A substrate concentration of 10 mM methylglyoxal was employed in the assay. When using intact cells, it must be taken into consideration that the glyoxal oxidase concentration is low and that the reaction must therefore be allowed to proceed longer. Thus, for example, very good readings were obtained after incubation for 9 hours. At a concentration of 1 mM methylglyoxal, no reaction was observed in the given window. Addition of 100 mM methylglyoxal only resulted in a slightly increased conversion rate, while the increase in the conversion rate from 2 mM to 10 mM methylglyoxal is within the linear part of the kinetics (FIG. 13).

### Example 11

### Enzyme Assay for Identifying Inhibitors

The enzyme assay was carried out in a total volume of 50 μl. To this end, the substances to be assayed were introduced in 10 μl substrate solution (50 mM methylglyoxal, 2.5% (v/v) DMSO) into a 384 microtitre plate. The K_{M} value of glyoxal oxidase for methylglyoxal had previously been determined (cf. FIG. 14). The concentration of the candidate compounds to be tested for an inhibitory effect was such that the final concentration of the substances in the assay carried out was 10 μM. In the next step, 20 μl of cell solultion (cells of the overproducer strain Bay-CA95 (OD₆₀₀=5); 0.2 M 2,2-dimethyl succinate buffer, pH 5, cooled at 4° C.) were added. 20 μl of detection solution (125 μM Amplex Red™ reagent (20 mM stock solution in 100% DMSO), 2.5 U/ml horseradish peroxidase, 62.5 mM sodium phosphate buffer, pH 7.4) were added to the mixture. The mixture was incubated for 9 hours at 30° C. Then, the increase in fluorescence was measured at λ=550 nm (absorption) and λ=595 nm (emission), the results of a measurement in the presence of Bay-CA95 cells being compared with the results of a measurement in the presence of the wild-type U. maydis 518 cells (see also FIG. 15). The substances used in the assay were present in the following final concentrations: c(2,2-dimethyl succinate/NaOH)=40 mM, c(Amplex Red® (Molecular Probes))=50 μM, c(horseradish peroxidase)=0.001 U/μl, c(methylglyoxal)=10 mM, OD (Bay-CA95)=1, c(sodium phosphate buffer)=25 mM. The inhibitory effect of a candidate compound could be seen from the decrease in relative fluorescence, and inhibitors were identified. Table II shows examples of compounds which act as glyoxal oxidase inhibitors. Table II also gives pI50 values which have been determined for the individual compounds. The pI50 value is the negative decimal logarithm of what is known as the IC50 value, which indicates the molar concentration of a substance which leads to 50% inhibition of the enzyme. For example, a pI50 value of 8 corresponds to half the maximum inhibition of the enzyme at a concentration of 10 nM. FIG. 15 shows an example of the effect of a compound (Tab. II, Example 3) on the activity of glyoxal oxidase.

TABLE II

| Example | Structural formula | pI50 |
|---|---|---|
| 1 | 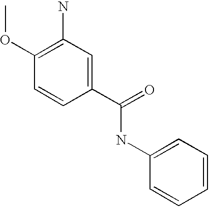 | 4.96 |
| 2 | 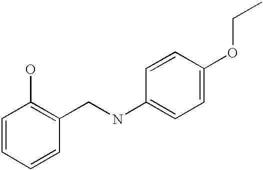 | 5.39 |
| 3 | 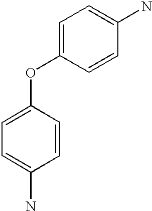 | 5.25 |
| 4 | 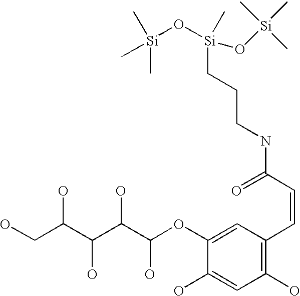 | 5.42 |
| 5 | 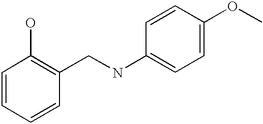 | 5.4 |

### Example 12

### Demonstration of the Fungicidal Effect of the Glyoxal Oxidase Inhibitors Which Have Been Identified

The antifungal action of the compounds (protective action) was tested, inter alia, on Venturia inaequalis as an example. This fungus causes what is known as apple scab, which leads to black and green mottled leaves in pomaceous fruit trees. The lesions enlarge and coalesce. Leaves which are severely infested die, which may lead to the trees losing all their leaves in summer. The infection also has an adverse effect on fruit set. Scab on fruits manifests itself in grey lesions on the skin, with suberification and deformed fruits.

To prepare a suitable preparation of active compound, 1 part by weight of active compound is mixed with, for example, 24.5 parts by weight of acetone and 24.5 parts by weight of dimethylformamide and 1.0 part by weight of alkylaryl polyglycol ether as emulsifier, and the concentrate is diluted with water to the desired concentration.

To test for protective activity, young plants are sprayed with the preparation of the active compound at the application rate stated. After the spray coating has dried on, the plants are inoculated with an aqueous conidial suspension of the apple scab pathogen Venturia inaequalis and then remain in an incubation cabinet for 1 day at approximately 20° C. and 100% relative atmospheric humidity.

The plants are then placed in a greenhouse at approximately 21° C. and a relative atmospheric humidity of approximately 90%.

1 to 12 days post-inoculation, the test is evaluated. 0% means an efficacy which corresponds to that of the control, while an efficacy of 100% means that no disease is observed.

At a concentration of 250 ppm, the compound of Example 4 (Tab. I) showed an efficacy of 45%.

### FIGURES AND SEQUENCE LISTING

FIG. 1

Determination of the homology between the U. maydis glyoxal oxidases Glo1, Glo2 and Glo3 according to the invention as shown in SEQ ID NO: 1 and SEQ ID NO: 3, the B. cinerea glyoxal oxidase and the known Phanerochaete chrysosporium glyoxal oxidase (BESTFIT). The similarity of U. maydis Glo1 and the P. chrysosporium gyloxal oxidase is 44%, while the identity is 38%. The conserved positions which are of importance for the coordination of the copper ion are shown against a grey background.

FIG. 2

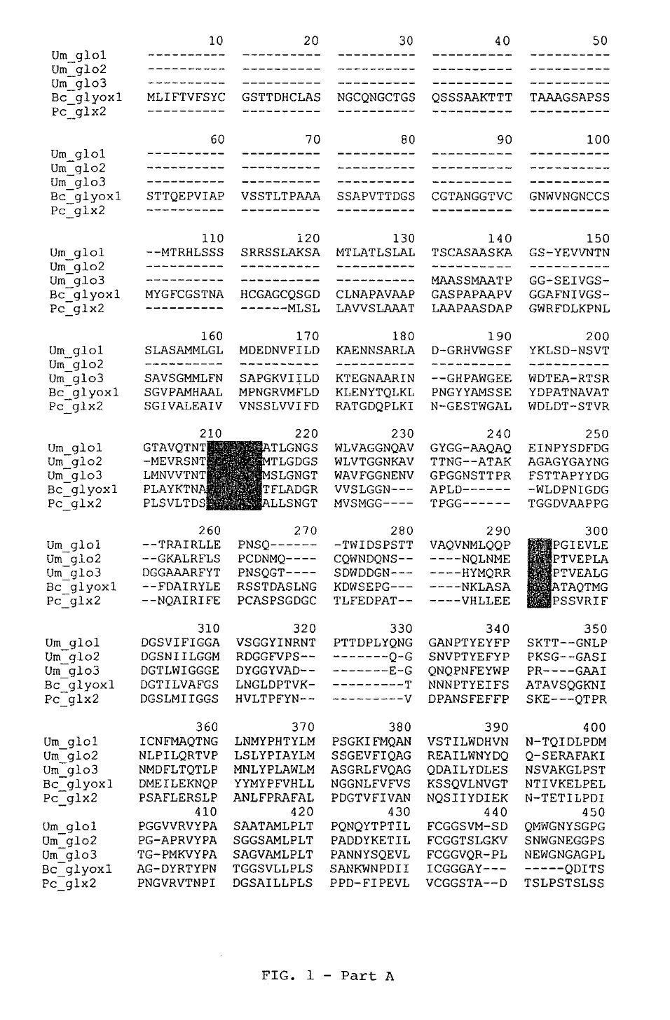

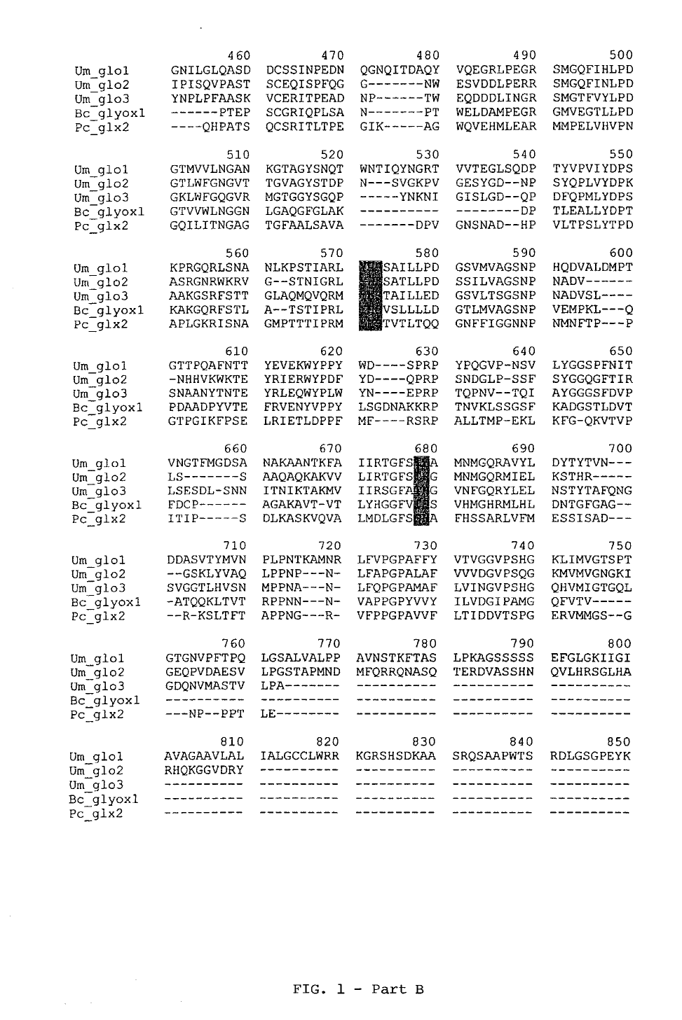

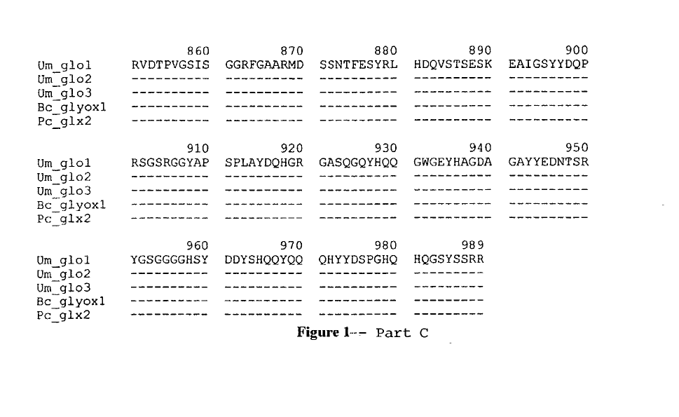

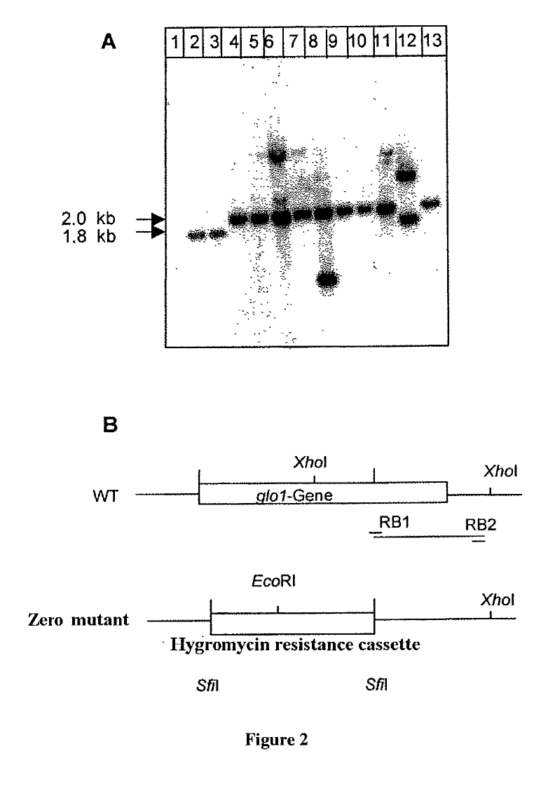

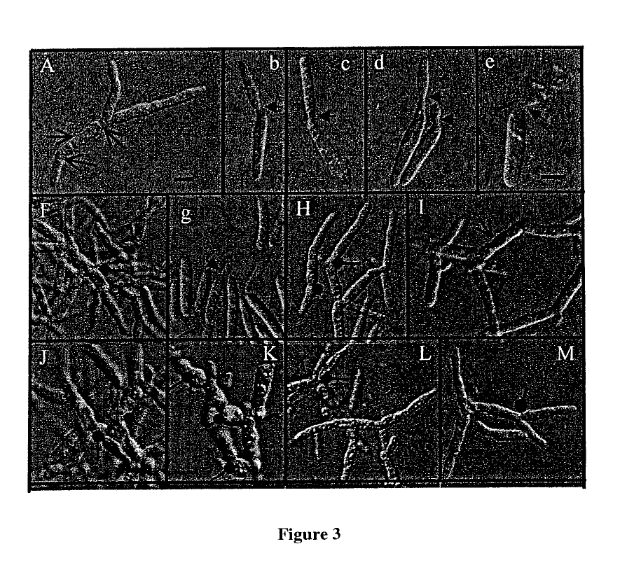

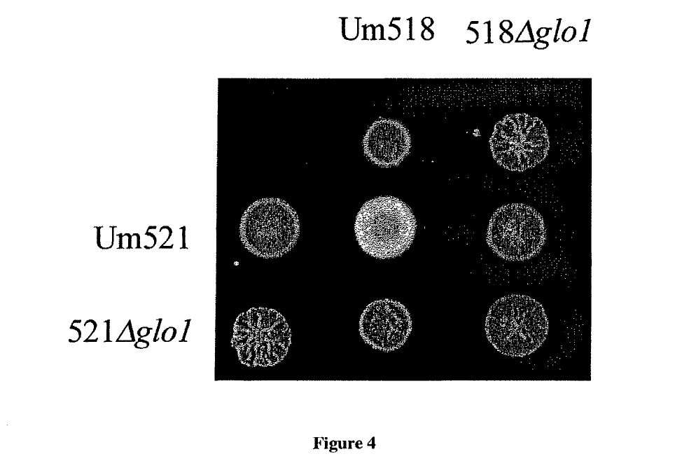

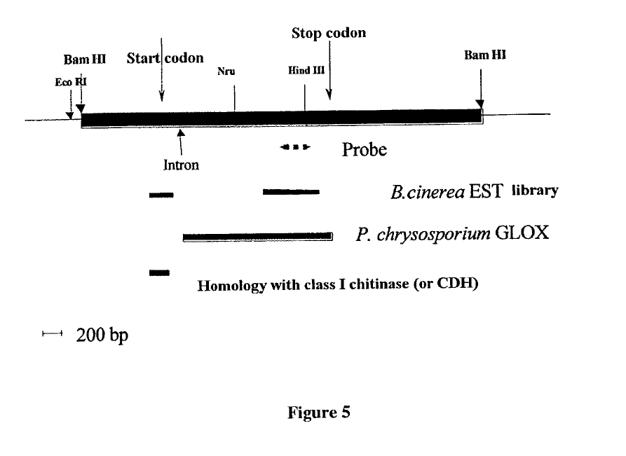

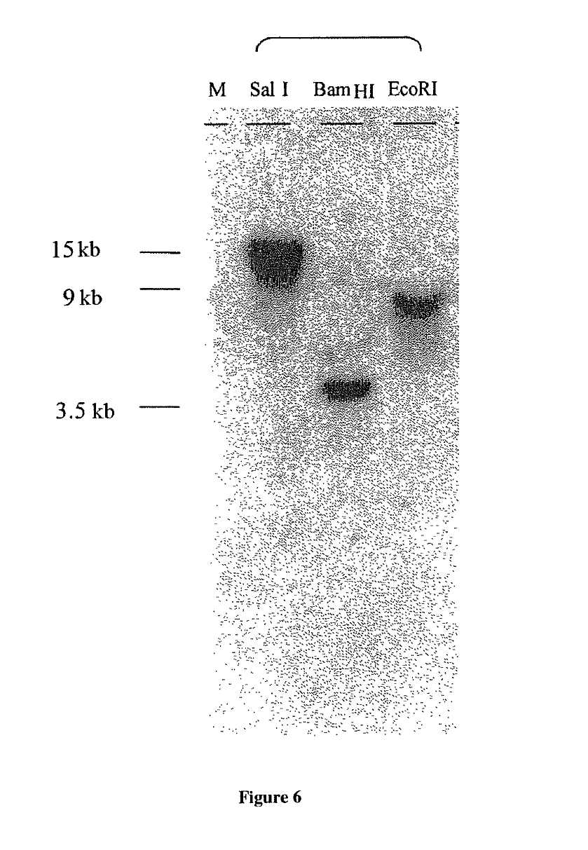

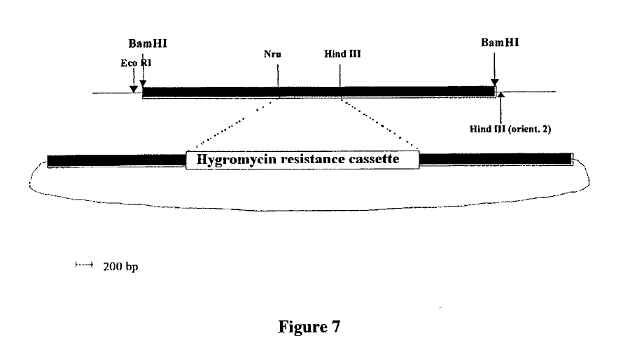

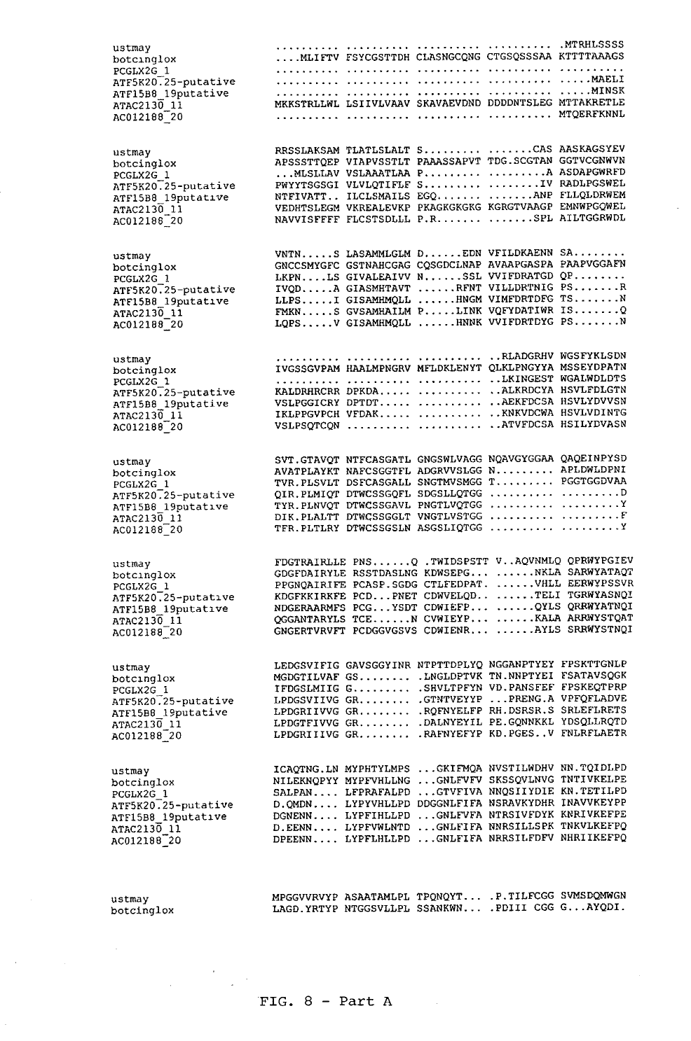

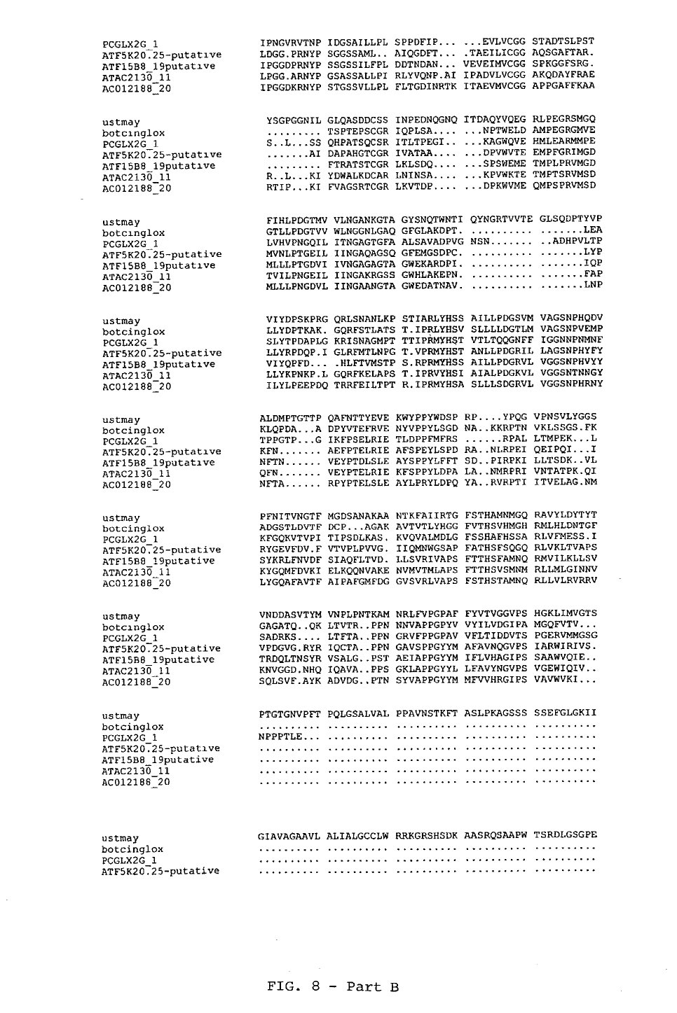

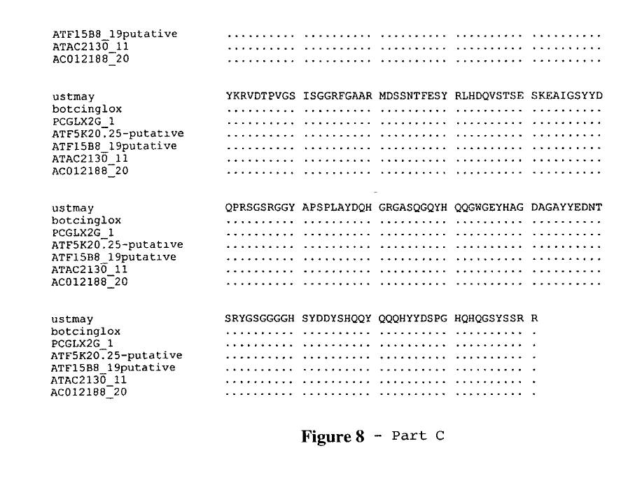

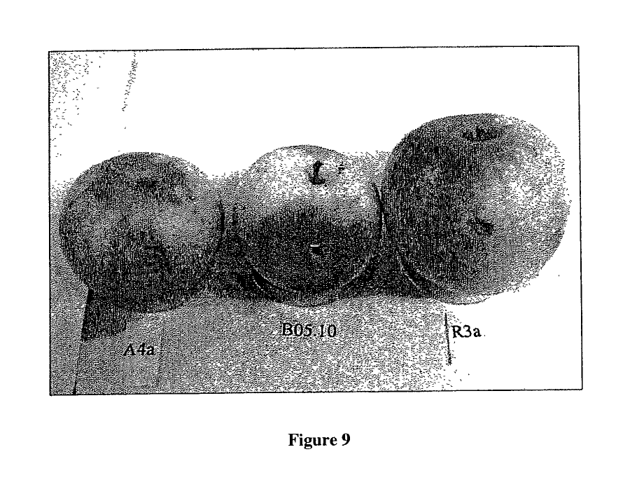

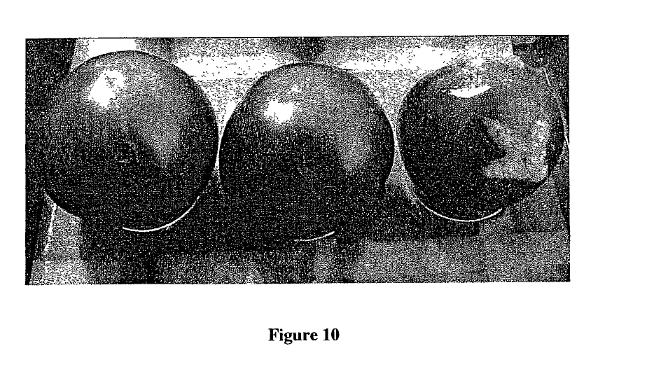

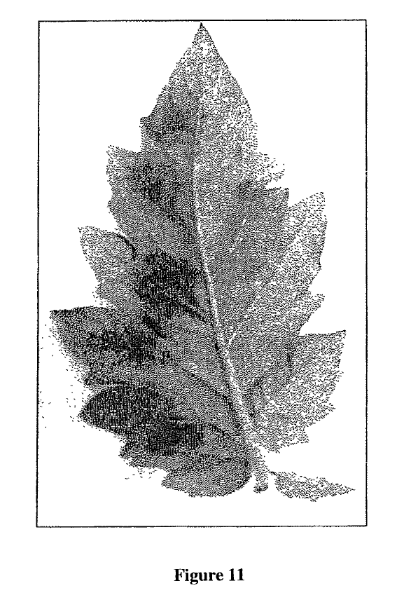

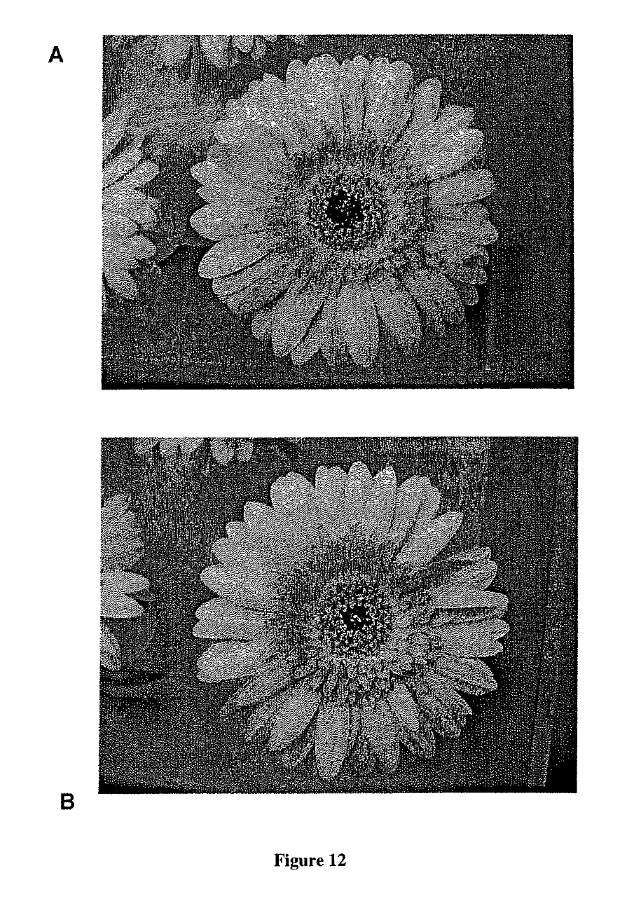

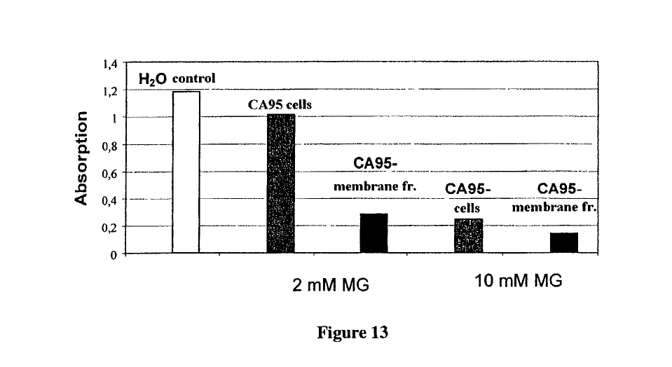

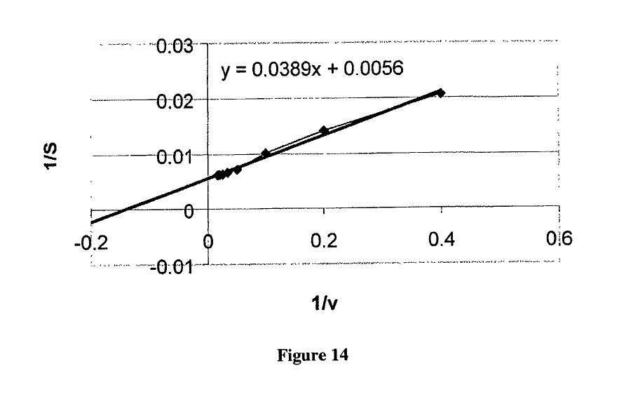

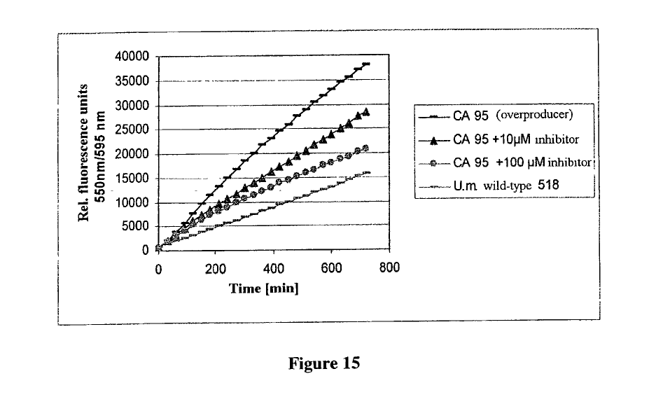

(A) Southern analysis for identifying glo1 zero mutants. 1 μg of genomic DNA of each of the Ustilago strains stated in each case was cut with EcoRI and XhoI, separated in a 1% agarose gel and blotted. Hybridization was effected with a digoxigenin-labelled DNA probe (1200 bp; PCR fragment with primers RB1/RB2 as shown in FIG. 2B). The DNA applied in the individual lanes was isolated from the following strains:

Lane 2: wild-type Um 518; lane 3: wild-type Um 521; lanes 4-8: transformants of Um 518 (518#0, 518#1, 518#4, 518#6, 518#8); lanes 9-13: transformants of Um 521 (521#1, 521#5, 521#7, 521#8, 521#9). The 1 kb plus DNA marker in lane 1 acted as size marker.

(B) Schematic representation of the homologous recombination for generating glo1 zero mutants. The primers RB1 and RB2 define the PCR product used as DNA probe for the hybridization (see also Kämper and Schreier (2001)).

FIG. 3

glo1 zero mutants show a pleiotropic morphology defect. The cultures in question were grown in PD medium to an OD₆₀₀ of 0.8, washed in H₂O and subsequently resuspended in a 0.2% Kelzan (Bayer AG) solution. Capital letters indicate zero mutants, while lower case letters indicate wild-types. A, b, c, F, G, J and K are Um518 strains or their derivatives; c, d, e, H, J, L and M are Um521 strains and their derivatives.

[symbol] : Bud necks in wild-type cells; [symbol] : additional septa; [symbol] : Y compounds, no cytokinesis; [symbol] : cells with rounded morphology. Also notable are the high degree of vacuolization, and the elongation and deformation of the mutant cells. The size marker shown corresponds to 3 μm.

FIG. 4

Phenotype of the (Delta)glo1 strains. The (Delta)glo1 allele was introduced into the U. maydis strains Um521 (alb1) and Um518 (a2B2). All of the strains, either alone or in the combinations stated, were applied dropwise to PD charcoal plate media. After incubation for 48 hours, the presence of a white aerial mycelium indicates successful mating.

FIG. 5

The main characteristics of the B. cinerea BcGlyox1 sequence. The protein sequence of BcGLYOX1 contains a putative signal peptide cleavage site followed by a short sequence with homology with a polysaccharide binding domain which can be found in plant proteins (for example in type I chitinases, lectins). This domain precedes the catalytic domain, which has homology with the P. chrysosporium gene encoding glyoxal oxidase and with the gene encoding galactose oxidases (from Dactylium dendroides). The BcGlyox1 gene also contains the unusual Cu²⁺ binding site, which is typical for the P. chrysosporium glyoxal oxidase. The cleavage sites used for isolating the gene are also shown. An intron which was found was marked, as was the position of the B. cinerea fragment used for the isolation and the DNA probe used for the Southern analysis.

FIG. 6

Southern blot with genomic DNA of B. cinerea (strain B05.10) cut with three different restriction enzymes as shown in the figure. The restricted DNA was hybridized with a radiolabelled 385 bp fragment from B. cinerea.

FIG. 7

Preparation of the vector pHyGLYOX1 used for generating knock-out mutants and containing a hygromycin-resistance cassette which replaces an NruI-HindIII fragment of the original vector.

FIG. 8

Sequence alignment between the sequences or sequence fragment encoding glyoxal oxidase from Ustilago maydis (Ustmay), Botrytis cinerea (botcinglox), Phanerochaete chrysosporium (PCGLX2G_{—}1) and various putative ORFs (encoding glyoxal oxidase) from Arabidopsis thaliana (ATF5K20.25-putative, ATF15B8_{—}19putative, ATAC2130_{—}11, AC012188_{—}20). Conserved amino acids of interest are shown against a grey background by way of example.

FIG. 9

Apathogenicity of the Knock-out Mutants

Excised apples (Alkmene and Cox Orange) were inoculated with a suspension of B. cinerea conidia (see Example 9). The inoculated host tissue was inoculated at 20° C. in the light. The BcGlyox1 mutants (knock-out mutants) which were tested were not capable of causing primary necrotic lesions (FIG. 9, A4a and R3a), while the wild-type caused primary lesions (FIG. 9, B05.10), which spread to some extent to the neighbouring tissue. In the case of the suspensions preincubated with malt extract (cf. Example 9), it also emerged that the mutants are not capable of infecting the test tissues.

FIG. 10

Apathogenicity of the Knock-out Mutants

Excised tomatoes (Lycopericon esculentum) were inoculated with a suspension of B. cinerea conidia (see Example 9). The inoculated host tissue was incubated at 15° C. in the dark. The BcGlyox1 mutants (knock-out mutants) which were tested were not capable of causing primary necrotic lesions (FIG. 10, tomato on the left, A4a, and in the middle, R3a), while the wild-type B05.10 caused primary lesions (FIG. 12, tomato on the right), which spread to some extent into the neighbouring tissue.

FIG. 11

Apathogenicity of the Knock-out Mutants

An excised tomato (Lycopericon esculentum) leaf was inoculated on one side in each case with a suspension of B. cinerea conidia (see Example 9). The inoculated host tissue was incubated at 1 5° C. in the dark. The BcGlyox1 mutants (knock-out mutants) which had been tested were not capable of causing primary necrotic lesions (FIG. 11, right half of the leaf), while the wild-type caused primary lesions (FIG. 11, left half of the leaf) which spread into the neighbouring tissue.

FIG. 12

Apathogenicity of the Knock-out Mutants

The excised flowers of gerbera hybrids were dusted with dry B. cinerea conidia (see Example 9). The inoculated host tissue was incubated at 15° C. in the dark. In all experimental set-ups, the BcGlyox1 mutants which were tested were not capable of causing primary necrotic lesions (FIG. 12A), while the wild-type caused primary lesions which spread to some extent into the neighbouring tissue (FIG. 12B).

FIG. 13

Comparison of the Conversion of Methylglyoxal by Glyoxal Oxidase as a Function of Different Substrate Concentrations

The expression of Glo1 was detected (cf. Example 10) in intact cells on the basis of the enzymatic conversion of methylglyoxal (MG) in CA95 cells (U. maydis strain BAY-CA95, cf. Example 3), in which Glo1 is overproduced. A substrate concentration of 10 mM methylglyoxal is employed in the test. At a concentration of 1 mM methylglyoxal, no reaction was observed in the given window. Addition of 100 mM methylglyoxal only resulted in a slightly increased conversion rate, while the increase in the conversion rate from 2 mM to 10 mM methylglyoxal is within the linear range of the kinetics. The test was carried out not only with intact cells, but also on cell fragments (membrane fraction).

FIG. 14

Lineweaver-Burk Plot for Determining the K_{M} of Glyoxal Oxidase for Methylglyoxal

The assay was carried out continuously by coupling the reaction with horseradish peroxidase (cf. Example 10). The conversion of Amplex Red® (molecular probes) was monitored fluorimetrically (·(exc)=550 nm; ·(em)=595 nm). The reaction volume was 50 μl. The conversion rate was determined after an incubation period of approximately 180 minutes (lag phase) and after deducting the blank value.

FIG. 15

Inhibition of Glo1 by Addition of an Inhibitor According to the Invention

The Glo1 activity was carried out using a coupled assay system with the detection reagent Amplex Red® as described in Example 10. Instead of Bay-CA95 cells (CA95) U. maydis wild-type 518 cells were used as control. One of the compounds identified in the method according to the invention (Tab. II, Example 3) (inhibitor) was employed in two different concentrations of 10 μM and 100 μM.

### SEQ ID NO: 1

Nucleic acid sequence encoding the U. maydis glyoxal oxidase Glo1 (cDNA).

### SEQ ID NO: 2

Amino acid sequence of the U. maydis glyoxal oxidase Glo1 encoded by the sequence as shown in SEQ ID NO: 1.

### SEQ ID NO: 3

Nucleic acid sequence encoding the U. maydis glyoxal oxidase Glo1 (genomic DNA).

### SEQ ID NO: 4

Amino acid sequence of the U. maydis glyoxal oxidase Glo1 encoded by the sequence as shown in SEQ ID NO: 3.

### SEQ ID NO: 5

Nucleic acid sequence encoding the U. maydis glyoxal oxidase Glo2 (cDNA).

### SEQ ID NO: 6

Amino acid sequence of the U. maydis glyoxal oxidase Glo2 encoded by the sequence as shown in SEQ ID NO: 5.

### SEQ ID NO: 7

Nucleic acid sequence encoding the U. maydis glyoxal oxidase Glo3 (cDNA).

### SEQ ID NO: 8

Amino acid sequence of the U. maydis glyoxal oxidase Glo3 encoded by the sequence as shown in SEQ ID NO: 7.

### SEQ ID NO: 9

Nucleic acid sequence encoding the B. cinerea glyoxal oxidase (cDNA).

### SEQ ID NO: 10

Amino acid sequence of the aus B. cinerea glyoxal oxidase encoded by the sequence as shown in SEQ ID NO: 9.

### SEQ ID NO: 11

Nucleic acid sequence encoding the B. cinerea glyoxal oxidase (genomic DNA containing two exons, exon 1 and exon 2, and an intron).

### SEQ ID NO: 12

Amino acid sequence of the B. cinerea glyoxal oxidase encoded by the sequence as shown in SEQ ID NO: 11 (exons 1 and 2 were linked in this listing).

### References

1. Altschul, S. F., Madden, T. L., Schaffer, A. A., Zhang, J. Z.; Miller W. and Lipman, D. J. (1997), Gapped BLAST und PSI-BLAST generation of protein database search programs. Nucleic Acids Res. 25: 3389-3402.

2. Benito, E. P., ten Have, A., van't Klooster, J. W. and van Kan, J. A. L. (1998), Fungal and plant gene expression during synchronized infection of tomato leaves by Botrytis cinerea. European Journal Plant Pathology 104, 207-200.

3. Bevan, M. (1984), Binary Agrobacterium vectors for plant transformation. Nucleic Acids Res 12(22): 8711-8721.

4. Bevan, M., Flavell, R. B., and Chilton, M.-D. (1983), A chimeric antibiotic resistance gene as a selectable marker for plant cell transformation. Nature 304, 184.

5. Bölker, M., Böhnert, H. U., Braun, K -H., Görl, J. and Kahmann, R. (1995), Tagging pathogenicity genes in Ustilago maydis by restriction enzyme-mediated integration (REMI). Mol. Gen. Genet. 248: 547-552.

6. Bottin, A., Kämper, J., Kahmann, R. (1996), Isolation of a carbon source-regulated gene from Ustilago maydis. Mol. Gen. Genet. 253, 342-352.

7. Gietz R D, Triggs-Raine B, Robbins A, Graham K C, Woods R A. (1997), Identification of proteins that interact with a protein of interest: applications of the yeast two-hybrid system. Mol Cell Biochem. 172(1-2):67-79.

8. Gillissen, B., Bergemann, J., Sandmann, C. Schroeer, M., Bölker, M., and Kahmann, R. (1992), A two-component regulatory system for self/nonself recognition in Ustilago maydis. Cell, 68: 647-657.

9. Gruber, M. Y and Grosby, W. L. (1993) Meth. in Plant Mol. Biol. and Biotechnology (Glick, B. und Thompson, J, eds), CRC Press Inc., Boca Raton, Fla.

10. Holliday, R. (1974), Ustilago maydis. In King, R. C. (ed), Handbook of Genetics. Plenum, N.Y., pp. 575-595.

11. Hollomon, D. W. (1979), Specificity of ethirimol in relation to inhibition of the enzyme adenosine deaminase. Proc. Br. Crop Prot. Conf. Pests Dis., p.251.

12. Kahmann, R. and Basse, C. (1999), REMI and its impact on the isolation of pathogenicity genes in fungi attacking plants. Europ. J. Plant Pathology 105, 221-229.

13. Kämper and Schreier (2001), Verfahren zur Herstellung von Deletionsmutanten [Method for generating deletion mutants]; German Patent Application No. 10 133 926.3.

14. Keon, J. P. R., White, G. A. and Hargraves, J. A. (1991), Isolation, characterization and sequence of a gene conferring resistance to the systemic fungicide carboxin from the maize smut pathogen Ustilago maydis. Curr. Genet. 19, 475-481.

15. Kersten, J. P. (1990), Glyoxal oxidase of Phanerochaete chrysosporium: its characterization and activation by lignin, Proc. Natl. Acad. Sci. USA 87 (8), 2936-2940.

16. Kersten J. P. and Cullen D. (1993): Cloning and Characterization of a cDNA encoding glyoxal oxidase, a H₂O₂-producing enzyme from the lignin-degrading basidiomycete Phanerochaete chrysosporium. PNAS (90), 7411-7413.

17. Kersten, J. P., Witek, C., Vanden Wymelenberg, A., Cullen, D. (1995) Phanerochaete chrysosporium Glyoxal Oxidase Is Encoded by Two Allelic Variants: Structure, Genomic Organization, and Heterologous Expression of glx1 and glx2, J. Bact. 172, 6106-6110.

18. Kersten, J. P. and Kirk, K. (1987): Involvement of a New Enzyme, Glyoxal Oxidase, in Extracellular H₂O₂ Production by Phanerochaete chrysosporium. J. Bact. 169, 2195-2201.

19. Lottspeich, F., Zorbas H. (ed.). (1998), Bioanalytik. Spektrum Akademischer Verlag, Heidelberg, Berlin.

20. Miki et al. (1993), Procedure for Introducing Foreign DNA into plants, in Methods in Plant Molecular Biology and Biotechnology (Glick, B. and Thompson, J., eds.), CRC Press Inc., Boca Raton, Fla.

21. Minet, M., Dufour, M. -E. and Lacroute, F. (1992), Complementation of S. cerevisiae auxotrophic mutants by A. thaliana cDNAs. Plant J. 2, 417-422.

22. Mumberg, D., Müller, R., Funk, M. (1995), Yeast vectors for the controlled expression of heterologous proteins in different genetic backgrounds. Gene 156, 119-122.

23. Sambrook, J., Fritsch, E. F. and Maniatis, T. (1989), Molecular cloning: A laboratory manual. Cold Spring Harbor Laboratory Press, Cold Spring Harbor, N.Y.

24. Schulz, B., Banuett, F., Dahl, M., Schlesinger, R., Schäfer, W., Martin, T., Herskowitz, I. and Kahmann, R. (1990), The b alleles of Ustilago maydis, whose combinations program pathogenic development, code for polypeptides containing a homeodomain-related motif. Cell 60, 295-306.

25. Spellig, T., Bölker, M., Lottspeich, F., Frank, R. W. and Kahmann, R. (1994): Pheromones trigger filamentous growth in Ustilago maydis. EMBO J., 13, 1620-1627.

26. Spellig, T., Bottin, A., Kahmann, R. (1996): Green fluorescent protein (GFP) as a new vital marker in the phytopathogenic fungus Ustilago maydis. Mol. Gen. Genet. 252, 503-509.

27. ten Have, Mulder, W., Visser, J. and van Kan, J. A. L. (1998), The endopolygalacturonase gene Bcpg1 is required for full virulence of Botrytis cinerea. Molecular Plant-Microbe Interactions 11, 1009-1016.

28. Thomson, J. D., Higgins, D. G. and Gibson, T. J., (1994), CLUSTAL W: Improving the sensitivity of a progressive multiple sequence alignment through sequence weighting, position specific gap penalties and weight matrix choice. Nucleic Acids Res., 22, 4673-4680.

29. Thornalley P. J., Jahan I., Ng R. (2001), Suppression of the Accumulation of Triosephosphates and Increased Formation of Methylglyoxal in Human Red Blood Cells during Hyperglycaemia by Thiamine in Vitro. J. Biochem. 129(4), 543-549.

30. Thornalley, J. P. (1996), Pharmacology of Methylglyoxal: Formation, Modification of Proteins and Nucleic Acids, and Enzymatic Detoxification: A Role in Pathogenesis and Antiproliferative Chemotherapy. Gen. Pharmac. 27(4), 565-563.

31. Tsukuda, T. et al. (1988) Isolation and characterization of an autonomously replicating sequence from Ustilago maydis. Mol. Cell. Biol. 8: 3703-3709.

32. Van der Vlugt-Bergmans, C. J. B., Wagemakers, C. A. M. and van Kan, J. A. L. (1997), Cloning and expression of the cutinase A gene of Botrytis cinerea. Molecular Plant-Microbe Interactions 10, 21-29.

33. Van der Vlugt-Bergmans, C. J. B., Brandwagt, B. F., Wagemakers, C. A. M., Van't Klooster, J. W. and van Kan, J. A. L. (1993), Genetic variation and segregation of DNA polymorphisms in Botrytis cinerea, Mycological Research 97, 1193-1200.

34. Van Kan, J. A. L., Van't Klooster, J. W., Dees, D. C. T., Wagemakers, C. A. M., and van der Vlugt-Bergmans, C. J. B. (1997), Cutinase A of Botrytis cinerea is expressed, but not essential, during penetration of gerbera and tomato. Molecular Plant-Microbe Interactions 10, 30-38.

35. Whittaker, M., Kersten, P. J., Cullen, D. and Whittaker, J. W. (1999), Identification of catalytic residues in Glyoxaloxidase by targeted mutagenesis. J. Biol. Chem. 274, 36226-36232.

36. Whittaker, M., Kersten, P. J., Nakamura, N., Sanders-Loehr, J. (1996) Glyoxal Oxidase from Phanerochaete chrysosporium is a New Radical-Copper Oxidase. J. Biol. Chem. 271(2) 681-687.

1. Method for identifying fungicides, characterized in that a chemical compound is tested in a glyoxal oxidase inhibition assay.

2. Method according to claim 1, characterized in that the fungicidal action of the compounds identified in the glyoxal oxidase inhibition assay are assayed on fungi.

3. Method according to claim 1, characterized in that fungal cells which express glyoxal oxidase are used in the glyoxal oxidase inhibition assay.

4. Nucleic acids encoding fungal polypeptides with the biological activity of a glyoxal oxidase, with the exception of the Phanerochaete chrysosporium sequences of Accession Nos: LM7286 and LM7287.

5. Nucleic acids according to claim 4, characterized in that they encode polypeptides from phytopathogenic fungi.

6. Nucleic acids according to claim 4 or 5, characterized in that they encode polypeptides from Basidiomycetes or Ascomycetes.

7. Nucleic acids according to claim 4, characterized in that they encode polypeptides from Ustilago and Botrytis.

8. Nucleic acids according to one of claims 4 to 7, characterized in that they take the form of the single-stranded or double-stranded DNA or RNA.

9. Nucleic acids according to one of claims 4 to 8, characterized in that they take the form of fragments of genomic DNA or the form of cDNA.

10. Nucleic acids according to one of claims 4 to 9 comprising a sequence selected from
a) a sequence as shown in SEQ ID NO: 1, SEQ ID NO: 3, SEQ ID NO: 5, SEQ ID NO: 7, SEQ ID NO: 9 and SEQ ID NO: 11,
b) sequences encoding a polypeptide which comprises an amino acid sequence as shown in SEQ ID NO: 2, SEQ ID NO: 4, SEQ ID NO: 6, SEQ ID NO: 8, SEQ ID NO: 10 or SEQ ID NO: 12,
c) sequences encoding a polypeptide which comprises the amino acids tyrosine 1, tyrosine 2, histidine 1, histidine 2 and cysteine which are suitable for Cu²⁺ coordination,
d) part-sequences of the sequences defined under a) to c) which are at least 14 base pairs in length,
e) sequences with 50% identity, particularly preferably 70% identity, very particularly preferably 90% identity, with the sequences defined under a) to c),
f) sequences which are complementary to the sequences defined under a) to c), and
g) sequences which, owing to the degeneracy of the genetic code, encode the same amino acid sequence as the sequences defined under a) to c).

11. DNA construct comprising a nucleic acid according to one of claims 4 to 10 and a heterologous or homologous promoter.

12. Vector comporising a nucleic acid according to one of claims 4 to 10, or a DNA construct according to claim 11.

13. Vector according to claim 12, characterized in that the nucleic acid is linked operably to regulatory sequences which ensure the expression of the nucleic acid in prokaryotic or eukaryotic cells.

14. Host cell containing a nucleic acid according to one of claims 4 to 10, a DNA construct according to claim 11 or a vector according to claim 12 or 13.

15. Host cell according to claim 14, characterized in that it takes the form of a prokaryotic cell.

16. Host cell according to claim 14, characterized in that it takes the form of a eukaryotic cell.

17. Ustilago maydis strain with the deposit number DSM 14 509.

18. Polypeptide with the biological activity of a glyoxal oxidase which is encoded by a nucleic acid according to one of claims 4 to 10.

19. Polypeptide according to claim 18, characterized in that it comprises an amino acid sequence which has at least 20% identity with the sequence as shown in SEQ ID NO: 2, SEQ ID NO: 4, SEQ ID NO: 6, SEQ ID NO: 8, SEQ ID NO: 10 or SEQ ID NO: 12.

20. Antibody which binds specifically to a polypeptide according to claim 18 or 19.

21. Method for generating a nucleic acid according to one of claims 4 to 10, comprising the following steps:
(a) full chemical synthesis in a manner known per se or
(b) chemical synthesis of oligonucleotides, labelling the oligonucleotides, hybridizing the oligonucleotides with DNA of a genomic library or cDNA library generated starting from genomic DNA or mRNA from fungal cells, selecting clones which contain the desired nucleic acid and isolating the hybridizing DNA from these clones, or
(c) chemical synthesis of oligonucleotides and amplification of target DNA by means of PCR.

22. Method for generating a polypeptide according to claim 18 or 19 comprising the steps
(a) culturing a host cell according to one of claims 14 to 16 under conditions which ensure the expression of nucleic acid according to one of claims 4 to 10, or
(b) expressing a nucleic acid according to one of claims 4 to 10 in an in vitro system, and
(c) obtaining the polypeptide from the cell, the culture medium or the in vitro system.

23. Method of finding a chemical compound which binds to a polypeptide according to claim 18 or 19 and/or modulates the activity of this polypeptide, comprising the following steps:
(a) bringing a host cell according to one of claims 14 to 16, cells of the strain according to claim 17 or a polypeptide according to claims 18 or 19 into contact with a chemical compound or a mixture of chemical compounds under conditions which permit the interaction of a chemical compound with the polypeptide, and
(b) determining the chemical compound which binds specifically to the polypeptide, and optionally
(c) determining the compound which influences the activity of the polypeptide.

24. Method of finding a compound which modifies the expression of polypeptides according to claim 18 or 19, comprising the following steps:
(a) bringing a host cell according to one of claims 14 to 16 or cells of the strain according to claim 17 into contact with a chemical compound or a mixture of chemical compounds,
(b) determining the polypeptide concentration, and
(c) identifying the compound which specifically influences the expression of the polypeptide.

25. Use of polypeptides with the biological activity of a fungal glyoxal oxidase, of nucleic acids encoding it, or of DNA constructs or host cells containing these nucleic acids for finding new fungicidal active compounds.

26. Use of fungal glyoxal oxidases, of nucleic acids encoding them, or of DNA constructs or host cells containing these nucleic acids in methods according to claim 23 or 24.

27. Use of a modulator of a polypeptide with the biological activity of a glyoxal oxidase as fungicide.

28. Use of a modulator of a polypeptide with the biological activity of a glyoxal oxidase for preparing compositions for the treatment of diseases caused by fungi which are pathogenic for animals or humans.

29. Fungicidally active substances found by means of a method according to claim 23 or 24.

30. Use of a nucleic acid according to one of claims 4 to 10, of a DNA construct according to claim 8 or of a vector according to claim 12 or 13 for generating transgenic plants and fungi.

31. Transgenic plants, plant parts, protoplasts, plant tissues or plant propagation materials, characterized in that, after introduction of a nucleic acid according to one of claims 4 to 10, a DNA construct according to claim 11 or a vector according to claim 18 or 19, the intracellular concentration of a polypeptide according to claim 15 or 16 is increased in comparison with the corresponding wild-type cells.

32. Transgenic fungi, fungal cells, fungal tissue, protoplasts, or fungal propagation materials, characterized in that, after introduction of a nucleic acid according to one of claims 4 to 10, a DNA construct according to claim 11 or a vector according to claim 12 or 13, the intracellular concentration of a polypeptide according to claims 18 or 19 is increased in comparison with the corresponding wild-type cells.

33. Plants, plant parts, plant tissue or plant propagation materials, characterized in that they contain a polypeptide according to claim 18 or 19 whose biological activity or expression pattern is modified in comparison with the corresponding endogenous polypeptides.

34. Fungi, fungal cells, fungal tissue or fungal propagation materials, characterized in that they contain a polypeptide according to claim 18 or 19 whose biological activity or expression pattern is modified in comparison with the corresponding endogenous polypeptides.

35. Method of generating plants, plant parts, protoplasts, plant tissues or plant propagation materials according to claim 33, characterized in that a nucleic acid according to one of claims 4 to 10 is modified by mutagenesis.

36. Method of generating fungi, fungal cells, fungal tissue, protoplasts or fungal propagation materials according to claim 34, characterized in that a nucleic acid according to one of claims 4 to 10 is modified by mutagenesis.

37. Method of inducing or increasing the resistance of plants to attack by pathogens, characterized in that the plants are brought into contact with fungi which are no longer capable of expressing a glyoxal oxidase.

38. Use of mutants of phytopathogenic fungi which are no longer capable of expressing glyoxal oxidase for inducing or increasing the resistance of plants.


SEQUENCE LISTING (12 sequences)

SEQ ID NO: 1 - DNA, 2589 bp, Ustilago maydis
  CDS (1)..(2589)
atgacgaggcacctctcctcatcctcgaggcgctcctcgctcgccaaaagcgccatgaccctcgcaaccctttctctcgccctaacctcgtgcgcatcggccgccagcaaggccggctcatacgaggttgtcaacaccaactcactcgcctcggccatgatgctcggtttaatggacgaggacaacgtctttattctcgacaaagctgaaaacaactcggctcgtctcgccgatggccgtcatgtctggggttctttctacaagctttccgacaattcggtcaccggcaccgccgtccagaccaacactttctgtgcctctggtgccaccttgggaaatggttcttggcttgtagctggcggcaaccaggccgtaggttacggtggcgctgcacaggcccaggagatcaacccctactcggacttcgacggaactagggcgattcgtctgctcgaacccaactcgcagacgtggatcgactcgcccagtacaactgtcgcacaggtcaacatgctccagcaaccccgttggtaccccggtatcgaggttcttgaagacggtagcgttatctttatcggaggtgccgtctcgggcggctacattaatcgcaacacgcctaccactgatcctctttaccagaatggaggcgctaaccccacctacgaatactttccctccaagaccaccggaaacctacccatctgtaactttatggctcagactaacggtctcaacatgtacccgcacacctacctcatgccctctggcaagatcttcatgcaggccaacgtcagtaccatcctctgggaccacgtcaacaacactcagatcgaccttcccgacatgcctggcggagtcgtgcgcgtctaccccgcctcggctgccactgccatgctgccactcactcctcagaatcagtacacacctaccatcctgttttgcggtggtagtgtcatgagcgaccagatgtggggcaactacagtggtcccggtggcaacattctcggtctccaagcctctgatgactgctcgtccatcaaccccgaggacaatcagggcaaccagatcactgacgctcagtacgtccaggaggggcggcttcccgaaggtcgttccatgggacagttcatccacctccctgacggtaccatggtcgtcctcaacggcgccaacaagggaactgccggctattcgaaccagacatggaacaccatccagtacaacggtcgcaccgtcgtcaccgaaggtctttcgcaggatcccacttacgttcccgtcatctatgacccgtccaagcccagaggtcagcgtctctccaatgctaatctcaagccttccaccattgctcgtctctaccactcgagcgctattttgctccccgatggttccgtcatggttgcaggttccaacccgcatcaggatgttgcgctcgacatgcccaccggcaccacgcctcaggctttcaacaccacctacgaggttgaaaagtggtaccctccttactgggactcgccacgcccttacccacagggcgtgcccaattcggtgctgtacggcggcagtcctttcaacattaccgtcaacggtacctttatgggtgactcggccaacgccaaggcagccaacaccaagtttgccatcattcgtaccggtttctccacccacgccatgaacatggggcagcgcgccgtctacctcgactacacctacaccgttaacgatgacgcctcggtcacctacatggtcaaccctttgcccaacactaaggctatgaaccgcctctttgtgcctggcccggccttcttctacgtcaccgtcggtggcgtgccaagccatggcaagctgatcatggtgggaacttcccccactggcactggcaacgtccccttcactcctcagctcgggtctgcactcgtcgcgcttccccctgctgtcaacagcaccaaattcacagcctccctccccaaggctggcagcagctcttcctccgagtttggcctcggcaagatcattggtatcgctgttgctggcgccgcagttttggccctcattgctctcggctgttgtctgtggaggcgcaagggcaggagccatagcgacaaggctgcctcgcgccagtcggctgccccttggaccagccgcgaccttggctcgggtcccgagtacaagcgtgtcgacactcctgtcggatccatcagcggtggtcgctttggggccgccaggatggacagctcgaatacgtttgagagctatcggttgcacgaccaggtcagcacgagcgaaagcaaggaggcgattggcagctactacgaccaacctcgcagcggcagccgtggcggctacgctcctagcccgctcgcctacgaccaacacggacgtggcgcctcgcaaggccagtaccaccagcaaggctggggcgaataccacgctggcgatgctggtgcatactacgaggacaacactagcaggtacggcagcggtggcggtggacacagctacgatgattactcgcaccagcaataccaacagcagcattactatgacagcccaggtcatcagcaccaaggaagctactctagtcgacgctaa

SEQ ID NO: 2 - protein, 862 aa, Ustilago maydis
MTRHLSSSSRRSSLAKSAMTLATLSLALTSCASAASKAGSYEVVNTNSLASAMMLGLMDEDNVFILDKAENNSARLADGRHVWGSFYKLSDNSVTGTAVQTNTFCASGATLGNGSWLVAGGNQAVGYGGAAQAQEINPYSDFDGTRAIRLLEPNSQTWIDSPSTTVAQVNMLQQPRWYPGIEVLEDGSVIFIGGAVSGGYINRNTPTTDPLYQNGGANPTYEYFPSKTTGNLPICNFMAQTNGLNMYPHTYLMPSGKIFMQANVSTILWDHVNNTQIDLPDMPGGVVRVYPASAATAMLPLTPQNQYTPTILFCGGSVMSDQMWGNYSGPGGNILGLQASDDCSSINPEDNQGNQITDAQYVQEGRLPEGRSMGQFIHLPDGTMVVLNGANKGTAGYSNQTWNTIQYNGRTVVTEGLSQDPTYVPVIYDPSKPRGQRLSNANLKPSTIARLYHSSAILLPDGSVMVAGSNPHQDVALDMPTGTTPQAFNTTYEVEKWYPPYWDSPRPYPQGVPNSVLYGGSPFNITVNGTFMGDSANAKAANTKFAIIRTGFSTHAMNMGQRAVYLDYTYTVNDDASVTYMVNPLPNTKAMNRLFVPGPAFFYVTVGGVPSHGKLIMVGTSPTGTGNVPFTPQLGSALVALPPAVNSTKFTASLPKAGSSSSSEFGLGKIIGIAVAGAAVLALIALGCCLWRRKGRSHSDKAASRQSAAPWTSRDLGSGPEYKRVDTPVGSISGGRFGAARMDSSNTFESYRLHDQVSTSESKEAIGSYYDQPRSGSRGGYAPSPLAYDQHGRGASQGQYHQQGWGEYHAGDAGAYYEDNTSRYGSGGGGHSYDDYSHQQYQQQHYYDSPGHQHQGSYSSRR

SEQ ID NO: 3 - DNA, 2923 bp, Ustilago maydis
  CDS (238)..(2823)
acgttccttctcccttttcctcgctttcaccactgcctcgacgttccttctttggcttctgcagttctgactgttgccactttttcgtcccctccgtctcgcctttgatttatcaccaccgcgcactcattggctgcggcgaattaccacgctttgggctcacgccatccatcgctcagccacatttccattcaatatcactgagctctgtcttccagaaaggatcgtttacacaccatgacgaggcacctctcctcatcctcgaggcgctcctcgctcgccaaaagcgccatgaccctcgcaaccctttctctcgccctaacctcgtgcgcatcggccgccagcaaggccggctcatacgaggttgtcaacaccaactcactcgcctcggccatgatgctcggtttaatggacgaggacaacgtctttattctcgacaaagctgaaaacaactcggctcgtctcgccgatggccgtcatgtctggggttctttctacaagctttccgacaattcggtcaccggcaccgccgtccagaccaacactttctgtgcctctggtgccaccttgggaaatggttcttggcttgtagctggcggcaaccaggccgtaggttacggtggcgctgcacaggcccaggagatcaacccctactcggacttcgacggaactagggcgattcgtctgctcgaacccaactcgcagacgtggatcgactcgcccagtacaactgtcgcacaggtcaacatgctccagcaaccccgttggtaccccggtatcgaggttcttgaagacggtagcgttatctttatcggaggtgccgtctcgggcggctacattaatcgcaacacgcctaccactgatcctctttaccagaatggaggcgctaaccccacctacgaatactttccctccaagaccaccggaaacctacccatctgtaactttatggctcagactaacggtctcaacatgtacccgcacacctacctcatgccctctggcaagatcttcatgcaggccaacgtcagtaccatcctctgggaccacgtcaacaacactcagatcgaccttcccgacatgcctggcggagtcgtgcgcgtctaccccgcctcggctgccactgccatgctgccactcactcctcagaatcagtacacacctaccatcctgttttgcggtggtagtgtcatgagcgaccagatgtggggcaactacagtggtcccggtggcaacattctcggtctccaagcctctgatgactgctcgtccatcaaccccgaggacaatcagggcaaccagatcactgacgctcagtacgtccaggaggggcggcttcccgaaggtcgttccatgggacagttcatccacctccctgacggtaccatggtcgtcctcaacggcgccaacaagggaactgccggctattcgaaccagacatggaacaccatccagtacaacggtcgcaccgtcgtcaccgaaggtctttcgcaggatcccacttacgttcccgtcatctatgacccgtccaagcccagaggtcagcgtctctccaatgctaatctcaagccttccaccattgctcgtctctaccactcgagcgctattttgctccccgatggttccgtcatggttgcaggttccaacccgcatcaggatgttgcgctcgacatgcccaccggcaccacgcctcaggctttcaacaccacctacgaggttgaaaagtggtaccctccttactgggactcgccacgcccttacccacagggcgtgcccaattcggtgctgtacggcggcagtcctttcaacattaccgtcaacggtacctttatgggtgactcggccaacgccaaggcagccaacaccaagtttgccatcattcgtaccggtttctccacccacgccatgaacatggggcagcgcgccgtctacctcgactacacctacaccgttaacgatgacgcctcggtcacctacatggtcaaccctttgcccaacactaaggctatgaaccgcctctttgtgcctggcccggccttcttctacgtcaccgtcggtggcgtgccaagccatggcaagctgatcatggtgggaacttcccccactggcactggcaacgtccccttcactcctcagctcgggtctgcactcgtcgcgcttccccctgctgtcaacagcaccaaattcacagcctccctccccaaggctggcagcagctcttcctccgagtttggcctcggcaagatcattggtatcgctgttgctggcgccgcagttttggccctcattgctctcggctgttgtctgtggaggcgcaagggcaggagccatagcgacaaggctgcctcgcgccagtcggctgccccttggaccagccgcgaccttggctcgggtcccgagtacaagcgtgtcgacactcctgtcggatccatcagcggtggtcgctttggggccgccaggatggacagctcgaatacgtttgagagctatcggttgcacgaccaggtcagcacgagcgaaagcaaggaggcgattggcagctactacgaccaacctcgcagcggcagccgtggcggctacgctcctagcccgctcgcctacgaccaacacggacgtggcgcctcgcaaggccagtaccaccagcaaggctggggcgaataccacgctggcgatgctggtgcatactacgaggacaacactagcaggtacggcagcggtggcggtggacacagctacgatgattactcgcaccagcaataccaacagcagcattactatgacagcccaggtcatcagcaccaaggaagctactctagtcgacgctaagccccgaaaaacgctgctggtgctttgtcagtcagtgcatgggggatcctctagagtcgacctgcaggcatgcaagcttggcactggccgtcgtttt

SEQ ID NO: 4 - protein, 862 aa, Ustilago maydis; identical to SEQ ID NO: 2

SEQ ID NO: 5 - DNA, 1614 bp, Ustilago maydis
  CDS (1)..(1614)
atggaggtgcgttccaacacgttctgtgccggcggtatgacgctgggcgacggcagttggctcgtcacgggcggaaacaaggcggttaccacgaatggcgcgactgctaaggcaggtgctggatacggcgcttacaatggcggtaaggcactgcgattccttagcccttgcgacaacatgcaatgtcagtggaacgaccaaaacagcaatcagctcaacatggagaggtggtatcctaccgtagagcctctagccgatggatccaatatcatccttggaggcatgcgcgacggtggctttgttccaagccagggctctaatgttcctacttacgagttctaccctcctaagagtggcggagctagtattaatttgccaatcctgcaacgtactgtacccctctcactctacccgatcgcgtatctcatgtcgtccggtgaggtgtttatccaagccggaagggaggcgatcctttggaattacgaccagcagagcgagcgcgcatttgccaagattccaggtgctcctcgtgtctatcctgcctctggtggctcggctatgcttcctctaactccggcagacgattacaaggagaccatcctcttctgcggtggtacgagcttgggcaaggtctcgaactggggtaacgagggtggaccctcgatccccatatctcaggttcccgcatcgacgtcgtgcgagcagatcagcccattccagggtggaaactgggaatcggtcgacgatttgcccgagcgtcgttccatgggtcaatttatcaacctgcccgacggcaccctgtggttcggcaacggtgtcaccactggcgttgctggttacagcaccgaccccaactctgtcggcaaaccggtgggcgagtcgtatggcgacaacccgtcgtaccagcctctcgtatacgaccccaaggcaagccgaggcaaccgatggaagcgcgtcggaagcaccaacattggtcgactctatcattcgtctgctacgctgcttccggattcgtctatcctcgttgctggttccaaccctaatgctgacgtcaaccaccatgtcaagtggaagacggaataccgcattgaacgatggtacccagacttctacgatcagcctcggccctcgaacgacggtctccctagctctttctcgtacggcggtcaaggctttaccatcaggctcagttctgcagcacaggcgcagaaggccaaggtggtcctgattcgaactggattttccacgcatggcatgaatatgggtcaacgcatgatcgagctcaagtcgacacatcggggcagcaagctctacgtagcgcagcttccacccaatccgaacctgtttgctcccggtcctgcgctcgcgttcgttgtagtcgatggcgttccgagtcaaggaaagatggtcatggtgggcaacggaaagatcggcgagcagcctgtcgatgcagagagcgtgctgcccggctcgaccgccccgatgaacgacatgtttcaaagacgacagaatgcgtcccagaccgaacgcgatgtggcttccagtcacaaccaagtgctccaccgaagcggcttgcatgcccgtcatcaaaagggtggcgtcgatcgttattga

SEQ ID NO: 6 - protein, 537 aa, Ustilago maydis
MEVRSNTFCAGGMTLGDGSWLVTGGNKAVTTNGATAKAGAGYGAYNGGKALRFLSPCDNMQCQWNDQNSNQLNMERWYPTVEPLADGSNIILGGMRDGGFVPSQGSNVPTYEFYPPKSGGASINLPILQRTVPLSLYPIAYLMSSGEVFIQAGREAILWNYDQQSERAFAKIPGAPRVYPASGGSAMLPLTPADDYKETILFCGGTSLGKVSNWGNEGGPSIPISQVPASTSCEQISPFQGGNWESVDDLPERRSMGQFINLPDGTLWFGNGVTTGVAGYSTDPNSVGKPVGESYGDNPSYQPLVYDPKASRGNRWKRVGSTNIGRLYHSSATLLPDSSILVAGSNPNADVNHHVKWKTEYRIERWYPDFYDQPRPSNDGLPSSFSYGGQGFTIRLSSAAQAQKAKVVLIRTGFSTHGMNMGQRMIELKSTHRGSKLYVAQLPPNPNLFAPGPALAFVVVDGVPSQGKMVMVGNGKIGEQPVDAESVLPGSTAPMNDMFQRRQNASQTERDVASSHNQVLHRSGLHARHQKGGVDRY

SEQ ID NO: 7 - DNA, 1902 bp, Ustilago maydis
  CDS (1)..(1902)
atggctgcatcgtccatggcggctacaccaggaggaagcgagatcgtcggctcgtccgccgtctcaggcatgatgctcttcaacagcgccccaggcaaagtcatcatcctcgacaagaccgaaggcaatgcagcccgcatcaacggccatcctgcttggggagaggagtgggacaccgaggctcgcaccagtcgtctgatgaacgtcgtcaccaacacgttttgtgcaggcggtatgtcgctcggcaacggcacctgggctgtctttggaggcaatgagaacgtcgggcccggaggcaactcgaccaccccacgtttcagcaccacagcgccttactatgatggcgatggaggcgctgctgctcgtttctacactcccaattctcagggcacctccgattgggatgatggtaaccactacatgcagaggcgcagatggtatccaactgtcgaagctctcggtgatggcacgctctggataggaggcggtgaagactatggaggttacgttgcagacgaaggacagaaccaacccaactttgagtactggccgccaagaggcgccgccatcaacatggactttcttacccagactttgccaatgaacctgtatcctttggcgtggctcatggcatccggtcgcttgtttgtccaggcagggcaggatgcgatcctgtacgacttggagagcaattcggttgccaaaggtcttccgtccaccacgggacccatgaaagtttacccggcttcagcgggcgtagctatgttgccactgacacccgcgaacaactattcgcaagaggtgctcttctgtggcggcgtgcagcgaccgcttaacgaatggggtaacggtgcgggtcctctgtacaacccacttccgtttgcggcaagcaaggtgtgcgagcgcatcacgcccgaggccgacaatccgacgtgggagcaggacgacgatctgatcaatggtcgatctatgggcacttttgtctatctgcccgacggaaagctgtggtttggacaaggggtgcgtatgggtaccgggggctattcaggtcagccttacaacaagaacattggtatttcgttgggcgaccaaccggacttccagccgatgctctacgatccttcagcggcgaagggctcgcgtttttcgacaactggcctagcgcagatgcaggtgcaaaggatgtaccattcgaccgccatcttgctcgaggacggctccgtgctcacttccggctccaaccctaacgccgacgtttcgcttagtaacgcagccaactacaccaacaccgagtaccgtctggagcagtggtacccgttgtggtacaacgagcccaggcctacgcagcccaacgtcactcagattgcttacggtggtggttcctttgacgtgccgctctctgaatcggacctctcgaacaacattaccaacatcaagacagccaagatggttattattcggtccggattcgcgacacacggtgtcaactttggacagcgctacctcgagctcaattcgacctacactgcctttcagaatggcagcgttggaggcacgctgcacgtgtccaacatgccgcctaacgctaaccttttccagcctgggccggccatggcatttttggtaatcaacggtgtgccttcccacggtcagcacgtaatgatcggcactggccagctgggcgaccagaatgtgatggcttcgacggtgcttcctgcctcacaggatccaccagcaccgagaacgggtagtagtggatctggctcgaaaggatccaacggctcgaatggatccaacggtactctgaaggactcgcccaatggtgccgttaccctgtcgacaggtctctgtgccagtgtatcctttgctgcagtgctgacggccttcgccctgtttgcttga

SEQ ID NO: 8 - protein, 633 aa, Ustilago maydis
MAASSMAATPGGSEIVGSSAVSGMMLFNSAPGKVIILDKTEGNAARINGHPAWGEEWDTEARTSRLMNVVTNTFCAGGMSLGNGTWAVFGGNENVGPGGNSTTPRFSTTAPYYDGDGGAAARFYTPNSQGTSDWDDGNHYMQRRRWYPTVEALGDGTLWIGGGEDYGGYVADEGQNQPNFEYWPPRGAAINMDFLTQTLPMNLYPLAWLMASGRLFVQAGQDAILYDLESNSVAKGLPSTTGPMKVYPASAGVAMLPLTPANNYSQEVLFCGGVQRPLNEWGNGAGPLYNPLPFAASKVCERITPEADNPTWEQDDDLINGRSMGTFVYLPDGKLWFGQGVRMGTGGYSGQPYNKNIGISLGDQPDFQPMLYDPSAAKGSRFSTTGLAQMQVQRMYHSTAILLEDGSVLTSGSNPNADVSLSNAANYTNTEYRLEQWYPLWYNEPRPTQPNVTQIAYGGGSFDVPLSESDLSNNITNIKTAKMVIIRSGFATHGVNFGQRYLELNSTYTAFQNGSVGGTLHVSNMPPNANLFQPGPAMAFLVINGVPSHGQHVMIGTGQLGDQNVMASTVLPASQDPPAPRTGSSGSGSKGSNGSNGSNGTLKDSPNGAVTLSTGLCASVSFAAVLTAFALFA

SEQ ID NO: 9 - DNA, 1970 bp, Botrytis cinerea
  CDS (1)..(1968)
atgctaatttttaccgtttttagttattgtggatctacaactgatcactgtttggcttccaatggttgccagaatggatgcacaggctcacaatcttcatcagccgccaagactactaccacagctgcagcaggcagcgcaccctcttcatctacaactcaagaaccagtgattgccccagttagttctacacttacgcctgccgcagctagcagtgcaccagtaactactgatggatcatgtggtactgccaatggaggtaccgtttgtggcaattgggtaaatggaaattgttgttccatgtacggtttttgtggcagtaccaatgcgcattgcggtgccggatgccaatcaggagattgtttgaatgcgcctgcggttgcagctcctggtgcaagccctgccccagctgccccagtaggaggtgcctttaatatcgtcgggtcgtctggagttcctgctatgcatgctgcacttatgccaaacggtcgagttatgttcctcgacaaattagagaactacacccaattgaaattgccaaatggatactacgccatgtcttcagaatacgacccagccactaacgcagtcgccactcctttagcttacaaaacaaatgcgttttgttccggaggcactttccttgctgatggacgtgttgtttctcttggaggcaacgcgcctttagattggctcgatccaaacattggggatggatttgacgccattagatatcttgaacgatcatctaccgatgctagcctcaatggaaaagactggagtgaaccaggtaacaagctcgcgagtgctcgttggtatgctactgctcaaactatgggtgatggaaccattttggtcgcttttggaagtttgaacggcctcgatccgactgtcaaaacgaacaacaatcctacatacgagattttcagtgctaccgctgtgtcgcaaggtaagaacattgacatggaaattttggagaaaaatcagccatattatatgtatccttttgttcatctcctcaatggtggaaatttgttcgtcttcgtttccaagtcttcccaagtactcaatgtcggtaccaacactatcgtcaaggaattacctgaacttgctggagactatcgcacatatcccaacactggtggaagtgttttactccctttgtcaagcgcaaacaaatggaaccctgatatcatcatctgcgggggaggtgcatatcaagatattaccagtccaacagagccaagttgtggaagaatccagccattgagtgcaaaccccacatgggagttggacgctatgcctgaaggccgtggtatggttgaaggaaccttacttccagatggaacagttgtctggcttaatggagggaacttgggtgctcaaggatttggacttgcaaaagacccaacattggaagctcttctttacgatcctacgaaagctaagggtcaaagattctcaactcttgcaacatcaactatcccacgtctctaccattctgtctctctcctccttcttgacggtacactaatggtcgctggctcaaaccctgtcgagatgccaaagcttcaaccagatgcagccgatccatatgttacggagttccgagttgagaactatgttcctccctatctctcaggcgataatgcaaagaagcgtcctactaatgtaaaattgtcatcaggtagcttcaaagcagatggtagcacacttgatgtcacatttgattgtccagctggcgcgaaagcagttactgtaactttgtaccacggtggattcgtcactcactctgtacatatgggtcatcgcatgctgcacttggataacacaggcttcggcgctggtgccacacagcagaagttgactgttactcgaccaccaaacaacaatgttgcacctccaggtccatatgttgtttacattcttgtagacggcattcctgccatgggacagtttgttacggtttg

SEQ ID NO: 10 - protein, 656 aa, Botrytis cinerea
MLIFTVFSYCGSTTDHCLASNGCQNGCTGSQSSSAAKTTTTAAAGSAPSSSTTQEPVIAPVSSTLTPAAASSAPVTTDGSCGTANGGTVCGNWVNGNCCSMYGFCGSTNAHCGAGCQSGDCLNAPAVAAPGASPAPAAPVGGAFNIVGSSGVPAMHAALMPNGRVMFLDKLENYTQLKLPNGYYAMSSEYDPATNAVATPLAYKTNAFCSGGTFLADGRVVSLGGNAPLDWLDPNIGDGFDAIRYLERSSTDASLNGKDWSEPGNKLASARWYATAQTMGDGTILVAFGSLNGLDPTVKTNNNPTYEIFSATAVSQGKNIDMEILEKNQPYYMYPFVHLLNGGNLFVFVSKSSQVLNVGTNTIVKELPELAGDYRTYPNTGGSVLLPLSSANKWNPDIIICGGGAYQDITSPTEPSCGRIQPLSANPTWELDAMPEGRGMVEGTLLPDGTVVWLNGGNLGAQGFGLAKDPTLEALLYDPTKAKGQRFSTLATSTIPRLYHSVSLLLLDGTLMVAGSNPVEMPKLQPDAADPYVTEFRVENYVPPYLSGDNAKKRPTNVKLSSGSFKADGSTLDVTFDCPAGAKAVTVTLYHGGFVTHSVHMGHRMLHLDNTGFGAGATQQKLTVTRPPNNNVAPPGPYVVYILVDGIPAMGQFVTV

SEQ ID NO: 11 - DNA, 2024 bp, Botrytis cinerea
  CDS (1)..(315)
  CDS (370)..(2022)
atgctaatttttaccgtttttagttattgtggatctacaactgatcactgtttggcttccaatggttgccagaatggatgcacaggctcacaatcttcatcagccgccaagactactaccacagctgcagcaggcagcgcaccctcttcatctacaactcaagaaccagtgattgccccagttagttctacacttacgcctgccgcagctagcagtgcaccagtaactactgatggatcatgtggtactgccaatggaggtaccgtttgtggcaattgggtaaatggaaattgttgttccatgtacggtttttggtaagtgcaatcattcactcacccgcgaatcttcgataatctaacacaatgtagtggcagtaccaatgcgcattgcggtgccggatgccaatcaggagattgtttgaatgcgcctgcggttgcagctcctggtgcaagccctgccccagctgccccagtaggaggtgcctttaatatcgtcgggtcgtctggagttcctgctatgcatgctgcacttatgccaaacggtcgagttatgttcctcgacaaattagagaactacacccaattgaaattgccaaatggatactacgccatgtcttcagaatacgacccagccactaacgcagtcgccactcctttagcttacaaaacaaatgcgttttgttccggaggcactttccttgctgatggacgtgttgtttctcttggaggcaacgcgcctttagattggctcgatccaaacattggggatggatttgacgccattagatatcttgaacgatcatctaccgatgctagcctcaatggaaaagactggagtgaaccaggtaacaagctcgcgagtgctcgttggtatgctactgctcaaactatgggtgatggaaccattttggtcgcttttggaagtttgaacggcctcgatccgactgtcaaaacgaacaacaatcctacatacgagattttcagtgctaccgctgtgtcgcaaggtaagaacattgacatggaaattttggagaaaaatcagccatattatatgtatccttttgttcatctcctcaatggtggaaatttgttcgtcttcgtttccaagtcttcccaagtactcaatgtcggtaccaacactatcgtcaaggaattacctgaacttgctggagactatcgcacatatcccaacactggtggaagtgttttactccctttgtcaagcgcaaacaaatggaaccctgatatcatcatctgcgggggaggtgcatatcaagatattaccagtccaacagagccaagttgtggaagaatccagccattgagtgcaaaccccacatgggagttggacgctatgcctgaaggccgtggtatggttgaaggaaccttacttccagatggaacagttgtctggcttaatggagggaacttgggtgctcaaggatttggacttgcaaaagacccaacattggaagctcttctttacgatcctacgaaagctaagggtcaaagattctcaactcttgcaacatcaactatcccacgtctctaccattctgtctctctcctccttcttgacggtacactaatggtcgctggctcaaaccctgtcgagatgccaaagcttcaaccagatgcagccgatccatatgttacggagttccgagttgagaactatgttcctccctatctctcaggcgataatgcaaagaagcgtcctactaatgtaaaattgtcatcaggtagcttcaaagcagatggtagcacacttgatgtcacatttgattgtccagctggcgcgaaagcagttactgtaactttgtaccacggtggattcgtcactcactctgtacatatgggtcatcgcatgctgcacttggataacacaggcttcggcgctggtgccacacagcagaagttgactgttactcgaccaccaaacaacaatgttgcacctccaggtccatatgttgtttacattcttgtagacggcattcctgccatgggacagtttgttacggtttg

SEQ ID NO: 12 - protein, 656 aa, Botrytis cinerea; identical to SEQ ID NO: 10